(19) United States
(12) Patent Application Publication
(10) Pub. No.: US 2026/0348943 A1
(43) Pub. Date: Nov. 5, 2026

(54) AUTOMATED BIOCHIPS FOR PERSONALIZED CANCER SCREENING AND PREDICTIVE DRUG RESPONSE ANALYSIS

(71) Applicant: Encapsulate Inc., Farmington, CT (US)

(72) Inventor: Armin Tahmasbi Rad, Farmington, CT (US)

(21) Appl. No.: 19/553,977

(22) Filed: Mar. 2, 2026

Related U.S. Application Data
(60) Provisional application No. 63/799,079, filed on May 2, 2025.

(51) Int. Cl. G01N 33/50; B01L 3/00

(52) U.S. Cl. CPC G01N 33/5011; B01L 3/502715; G01N 33/5082; B01L 2200/16; B01L 2300/069; B01L 2300/0874

(57) ABSTRACT

Systems and methods for predictive drug response assessments are provided, in particular for growing tumor cells in a manner that mimics the native three-dimensional environment. Cell cultures grown in the devices or by the methods can be screened for inhibition by specific chemotherapeutics or other drugs. In general, the devices and methods can be used to evaluate the effects of different compositions and environments on a tumor.

### CROSS-REFERENCE TO RELATED APPLICATIONS

The present application claims priority from U.S. Provisional Patent Application No. 63/799,079, filed May 2, 2025, which application is incorporated herein by reference. The subject matter of this patent application may be related to subject matter of the following patents or patent applications, each of which is hereby incorporated herein by reference in its entirety: US 12,227,726 to Nieh et al.; US 11,760,966 to Nieh et al.; and WO 2020/252225 filed by the University of Connecticut.

### STATEMENT REGARDING FEDERALLY SPONSORED RESEARCH

This invention was partially made with government support under Grant No. NSF 2208230 awarded by the National Science Foundation.

### FIELD OF THE INVENTION

This invention relates to precision oncology, microfluidic biochips, and patient-derived tumor models for diagnostics, prognosis, and predictive drug response assessments. The inventions described and claimed herein relate to devices and methods for growing tumor microreplicates or microtumors in a manner that mimics the native three-dimensional environment. Tumor microreplicates grown in the devices or by the methods can be screened for inhibition by specific chemo-, targeted-, hormonal-, radio-, immuno-, and gene-therapy options or other therapeutics such as anti-body drugs, oncolytic virus treatments, nanoparticles, and microbiome-modulating. In general, the devices and methods can be used to evaluate the effects of different compositions and environments on a tumor.

### BACKGROUND OF THE INVENTION

Modern cancer treatment suffers from a fundamental mismatch between treatment protocols and individual tumor biology. Current standards rely on generalized regimens, with therapeutic decisions based largely on population data, genetic markers, and clinical heuristics rather than direct functional response data. This results in ineffective treatments, toxic side effects, prolonged suffering, and inflated costs.

Personalized cancer treatment, which tailors therapy to a patient's unique genetic makeup, tumor biology, and overall health, can improve outcomes by identifying the most effective treatments while reducing side effects. However, the heterogeneity of cancer presents a major challenge, as variations in genetic profiles, tumor biology, and treatment responses exist not only between different cancers but also within the same tumor. This intratumoral heterogeneity complicates the identification of the most effective therapies and necessitates innovative diagnostic tools.

To treat cancer, especially in advanced stages, oncologists often select a therapy based on its success rate in previous patients. If the first line of treatment fails, alternative drugs may be administered in multiple rounds of treatment. Patients undergoing chemotherapy or most of the other cancer treatment options often endure several treatment cycles before clinicians can determine efficacy. The reason for these failures has been attributed to the genetic variability in between patients, and the inherent heterogeneous nature of cancer, and its innate ability to acquire resistance against certain drugs. For example, 80% of advanced ovarian cancer patients encounter failure in their first cycle of therapy. This wait-and-see approach not only delays optimal care but also leads to the progression of disease.

Therefore, improved devices for screening the efficiency of therapies against a patient's cancer cells prior to starting treatment would greatly improve the success rates and would diminish complications.

### SUMMARY OF VARIOUS EMBODIMENTS

Described herein are three-dimensional (3D) multi-tumor-on-a-chip microfluidic devices to screen mechanical, physical, biological, chemical, and electrical cues, such as drugs, gravitational forces, or other stimulations, against certain types of cancer. The advantage of systems described herein is that the tumor cells are encapsulated in a hydrogel matrix that replicates the tissue environment of a subject's body. This provides a confined space and permits diffusion of various treatments (including, but not limited to: chemotherapies, targeted therapies, hormonal therapies, immunotherapy agents, growth factors, and radiotherapy exposures) in a manner similar to the natural three-dimensional environment of the organism. The system described herein can also be used as a research tool for investigating new treatment methods on a variety of cell or tumor types. The system can also be used for evaluating impact of external conditions, such as electrical, mechanical, chemical, or biological cues, such as zero- or micro- gravity. The device or system includes:
a single step tissue-on-a-chip platform to be used in clinics, labs, and pharmaceutical research and development. The described process requires mixing the cells with curable or injectable hydrogels and introducing the combination to the chip. This delivers the cells to the chambers allowing continuous growth;
a multi-gel extracellular structure for mimicking the pharmacokinetics of drug delivery;
a mechanism that pre-defines the initial size, shape and location of the tumor, where the shape is defined by the cancer type mimicking the natural structure of the disease, with a clear boundary, enabling accurate monitoring and measuring of tumor size and growth;

In some embodiments, the invention further comprises a set of fabrication methodologies enabling the generation of discrete microtumor replicates (microreplicates) within the device using advanced hydrogel-based formation techniques. These methodologies include but are not limited to hydrogel-based tumoroid formation methods, droplet-based encapsulation, micro-molded hydrogel well arrays, and both sacrificial templating and subtractive manufacturing techniques. These methods allow for precise control over the three-dimensional architecture, spatial localization, and size uniformity of each microtumor core. For instance, droplet-based encapsulation enables the formation of high-throughput, size-controlled hydrogel spheres in which tumor cells are suspended and cured. In parallel, micro-molded wells provide templated confinement of hydrogel-cell mixtures, allowing consistent shape definition and cellular distribution across replicates.

In certain embodiments, sacrificial templating or subtractive manufacturing is used to generate the primary and secondary hydrogel boundaries, or perfusable voids or nutrient channels within the hydrogel scaffold prior to or after cell encapsulation. These channels simulate vascularization or facilitate the controlled delivery of nutrients, oxygen, or therapeutic agents to microtumor structures. For example, a sacrificial filament (e.g., gelatin or Pluronic F127) may be printed or embedded within a photo- or temperature- crosslinkable hydrogel matrix and subsequently removed to form hollow channels. Subtractive laser etching or enzymatic patterning may also be employed to sculpt microarchitectures that reflect in vivo tumor boundaries. These techniques collectively enhance the physiological relevance of the model and enable a higher degree of fidelity when modeling tumor growth, drug response, and resistance mechanisms within the chip platform.

a multilayer device fabrication allowing simple modifications to scale-up for various applications; and

a combination technique for multiple hydrogels that produce measurable predefined microtumor structures suitable for screening drugs.

In one embodiment, the first cured hydrogel comprises uncured spaces comprising one or more of cylindrical, spherical, cubic, or other shaped voids that can be occupied by a population of tumor cells, or tumor fragments (which can be referred to as bio-blobs), encapsulated in the second cured hydrogel. Accurate models of the tumor help predict individual responses to anti-cancer drugs, capturing the morphological, molecular, and functional characteristics of patient tumors.

In one aspect of the invention, the chip contains multiple “cores” for microtumor growth. Hydrogels and other environmental conditions, e.g. heat/temprature, nutrients, retardants, are adjusted to create standardized microtumors from the same patient samples. Since the microtumors are standardized, the evaluation process with respect to diagnostics, prognosis, and drug interaction is automated. The invention enables a “one-shot” personalized clinical trials because each microtumor core can be functionalized as a control, a no-drug intervention test (e.g. subject to nutrient and temperature manipulation), and a drug intervention test. Furthermore, multiple standard drug intervention cores enable application of various doses and combinations of drugs within the same evaluation process. As a result, the invention enables implementation of various logical combinations of no-drug and drug-based interventions so that results can be easily and reliably compared.

In one embodiment, a multi-layer microfluidic device, comprising a first unit having a media membrane and a first plurality of layers defining a first set of channels, and a second unit having a chamber layer and a second plurality of layers defining a second set of channels. The chamber layer defines a hydrogel chamber, the hydrogel chamber being adapted to receive a plurality of hydrogel-encapsulated tumoroids when the first and second units are not joined to each other, wherein the media membrane further defines the hydrogel chamber when the first and second units are joined together, the media membrane permitting the flow of a mixture of a media and a composition into the hydrogel chamber. The composition may include a chemotherapeutic agent and/or a growth factor. An imaging system is preferably provided to analyze the tumoroids in the hydrogel chamber over a period of time, recording changes in the tumoroid and the cells.

In a preferred embodiment, the microfluidic device further includes a reservoir defined by at least one of the first and second units, a mixing chamber defined by at least one of the first and second units, the mixing chamber being adapted to receive the media and the composition, mix the media and the composition, and permit the mixture to flow to the hydrogel chamber, and a cell-collection chamber defined by at least one of the first and second units. The cell-collection chamber collects cells shed by the tumoroids in the hydrogel chamber. An imaging system monitors these shed cells over a period of time. The microfluidic device also preferably includes a media-collection chamber defined by at least one of the first and second units, and a terminal collection chamber defined by at least one of the first and second units. The terminal collection chamber may simply collect waste from upstream chambers. Alternatively, the terminal collection chamber may also collect shed cells flowing from an upstream chamber. The imaging system may also analyze these shed cells in the terminal collection chamber.

In one embodiment, the device may include a first follow-on collection chamber downstream of the cell collection chamber and may further include a second follow-on collection chamber downstream of the first follow-on collection chamber. In such an embodiment, the cell collection chamber is preferably adapted to hold cells shed from the tumoroid within about the previous day, the first follow-on collection chamber is adapted to hold cells shed from the tumoroid from about one to five days earlier, the second follow-on collection chamber is adapted to hold cells shed from the tumoroid from more than about five days earlier. The imaging system may analyze these shed cells in the first and/or second follow-on collection chambers. The shed cells collected in the various chambers—the terminal collection chamber, the cell collection chamber, and/or a follow-on collection chamber—may indicate the propensity of the tumoroid to metastasize.

The hydrogel chamber preferably includes a first cured hydrogel providing a plurality of uncured spaces, each of the uncured spaces being capable of being filled with a hydrogel-encapsulated tumoroid. The first and second cured hydrogels may be the same material and may be cured by different mechanisms.

The cell-collection chamber is, in a preferred embodiment, located in a flow path between the hydrogel chamber and the terminal collection chamber, with the cell collection chamber being further defined by a cell-collection membrane. The cell-collection membrane prevents tumoroid cells from migrating from the hydrogel chamber to the terminal collection (or waste) chamber, so that the migrating tumoroid cells are collected in the cell-collection chamber. Preferably, the cell membrane and the media membrane are formed from different areas of a single membrane sheet.

A method for monitoring cell growth and viability, is also provided in one embodiment of the invention. The method comprises providing a device comprising: a first unit comprising a first plurality of layers defining a first set of channels. A second unit is also provided comprising a second plurality of layers defining a second set of channels, and a chamber layer defining a hydrogel chamber, the hydrogel chamber being adapted to receive a plurality of hydrogel-encapsulated tumoroids when the first and second units are not joined to each other, wherein the hydrogel chamber includes a first cured hydrogel providing a plurality of uncured spaces, each of the uncured spaces being capable of being filled with a tumoroid encapsulated in a second cured hydrogel. The method further includes isolating cells from a subject or a human or animal cell line; encapsulating the cells in the second curable hydrogel; inserting the hydrogel-encapsulated cells into at least one of the uncured spaces in the hydrogel chamber while the first and second units are not joined to each other; joining the first and second units together; providing a composition; causing the composition to flow into the hydrogel chamber and analyzing the hydrogel-encapsulated cells in the microfluidic device. The composition may comprise chemotherapeutic drugs, cytotoxins, antibiotics, anti-viral agents, immunomodulatory agents, and/or anti-inflammatory agents.

Preferably, the method further includes mixing a media and the composition in a mixing chamber defined by the device. The first unit preferably includes a media membrane that further defines the hydrogel chamber when the first and second units are joined together, the media membrane permitting the flow of a mixture of a media and a composition into the hydrogel chamber.

The device described above may also be used for screening the effectiveness of a composition on a tumor cell, by isolating cells from a subject or a human or animal cell line; providing in the hydrogel chamber a first hydrogel having uncured spaces while the first and second units are not joined to each other; encapsulating the cells in a second curable hydrogel; inserting hydrogel-encapsulated cells into the uncured spaces while the first and second units are not joined to each other; joining the first and second units together; providing a media; providing a composition; mixing the media and the composition in the mixing chamber; causing the mixture of the composition and media to flow through the media membrane into the hydrogel chamber and analyzing the hydrogel-encapsulated cells in the microfluidic device.

In one embodiment, a system is provided for identifying patient-specific optimal drug dosages, radiotherapy beam intensities, or other external cue exposure levels. The system includes at least one multi-layer microfluidic device, but preferably a plurality of multi-layer microfluidic devices is used surrounding a cell chamber. Each microfluidic devices includes a first inlet for introducing a dosage of a medication at a first concentration, and a second inlet for introducing a medium without any of the medication or the medication at a second concentration that is lower than the first concentration. The microfluidic device also includes a plurality of outlets, at least one outlet providing a concentration of the medication different from a concentration of another outlet. Between the inlets and the outlets, the microfluidic device includes an inlet-connecting channel fluidly connecting the first and second inlets, a plurality of inlet wells fluidly connected to the inlet-connecting channel, an outlet-connecting channel fluidly connecting the plurality of outlets, a plurality of outlet wells fluidly connected to the outlet-connecting channel, and an intermediate channel fluidly connected to at least one of the plurality of inlet wells or outlet wells. A microreplica chamber receives and contains cells to be exposed to different concentrations of medications. The cell chamber is fluidly connected to the plurality of outlets from each of the multi-layer microfluidic devices. When multiple microfluidic devices are used, the medication in each of multi-layer microfluidic devices may be the same, in order to test more gradations of concentration, but preferably different medications are used in each of the microfluidic devices. In a preferred embodiment, each multi-layer microfluidic devices further includes a plurality of intermediate wells fluidly connected to the intermediate channel. With this embodiment, precise gradients of concentrations can be produced to identify patient-specific optimal drug doses, down to minimum effective or resistance thresholds.

In another embodiment, a multi-layer microfluidic device is provided for evaluating therapies for a tumor. The device comprises an upper layer, a lower layer, and a middle layer. The middle layer is positioned between the upper layer and the lower layer and, when combined with the upper and lower layers, defines a cell chamber sized to hold an array of tumoroids. The cell chamber includes a hydrogel. Each of the upper, middle, and lower layers define at least five inlet channels and an outlet channel, wherein the inlet channels each provide a different medication. In a related embodiment, a method of evaluating therapies for a tumor. The method includes providing a microfluidic device comprising a plurality of layers, including a middle layer that, when combined with adjacent layers, defines a cell chamber in fluid communication with at least three inlet channels and an outlet channel, the cell chamber including a hydrogel, the inlet channels each providing a different medication. The method also includes placing an array of tumoroids in the cell chamber and exposing the array of tumoroids to different combinations of medication flowing from the inlet channels. The method also includes monitoring the reaction of the array of tumoroids to the different combinations of medications. In some implementations, the system is enabled to monitor microtumors continuously and/or at a predefined time pattern. The process of monitoring is automated and results in data collection according to pre-define parameters that can be correlated with microtumor growth conditions, drug injections, gravity changes and other variables. With this embodiment, different treatments can be mixed and applied in a variety of ratios, to assess toxicity synergistic effects of two to twelve drug combinations.

In another embodiment, a method is provided for evaluating the effects of different compositions on a tumor. The method includes providing a microfluidic device comprising a central cell chamber and a plurality of reservoirs surrounding the central cell chamber, each of the reservoirs being in fluid communication with the central cell chamber. The method also includes placing a pre-defined tumor in the central cell chamber, such that the tumor substantially fills the central cell chamber and is adjacent each of the reservoirs, and placing a different composition in each of the reservoirs. The compositions are allowed to interact with the tumor, and the reaction of the tumor to the different compositions is monitored. The microfluidic device may be tilted to affect the interactions between a composition and the tumor. The compositions may include a medicament, a potential carcinogen, or combinations thereof. With this embodiment, a pre-defined tumor can be monitored for growth and invasion towards the different preloaded compartments, indicating how certain materials such as growth factors, drugs, chemicals such as caffeine or tobacco, can activate tumors metastasis or increase cancer aggressiveness.

In another embodiment a multi-layer, multi-gel microfluidic device is provided having a plurality of inlets, outlets, and channels in fluid communication with each other. The microfluidic device includes a first middle layer comprising a cell-growing chamber in fluid communication with a culture medium channel. The cell-growing chamber is adapted to hold a population of tumor cells encapsulated in a cured hydrogel. The microfluidic device also includes a second middle layer comprising a hypoxic chamber positioned underneath the cell-growing chamber, such that the encapsulated tumor cells are exposed to a hypoxic environment. In a preferred embodiment, the cell-growing chamber further comprises a second cured hydrogel, wherein the second cured hydrogel provides a plurality of uncured spaces, the uncured space containing the cured hydrogel encapsulating the tumor cells.

In a related embodiment, a method is provided for evaluating the efficacy of a treatment (e.g., chemotherapy, radiotherapy) on a subject’s tumor. The method includes isolating tumor cells from a subject’s tumor, encapsulating the tumor cells in a curable hydrogel, inserting the cells in a multi-layer, multi-gel microfluidic device, incubating the encapsulated tumor cells in the microfluidic device under conditions favorable for tumor cell growth for a period of time, contacting the encapsulated tumor cells with one or more drugs for a period of time, analyzing changes or viability in the encapsulated tumor cells after the period of time, and evaluating the efficacy of the drug on the encapsulated tumor cells. In view of this evaluation, a treatment and/or dosage is initiated, maintained, or modified for the subject from whom the tumor cells were isolated. The microfluidic device includes a plurality of inlets, outlets, and channels in fluid communication, a first middle layer comprising a cell-growing chamber in fluid communication with a culture medium channel. The cell-growing chamber is adapted to contain a population of tumor cells encapsulated in a cured hydrogel. The microfluidic device also includes a second middle layer comprising a hypoxic chamber underneath the cell-growing chamber, such that the encapsulated tumor cells are exposed to a hypoxic environment. An example for such reservoir is growing microbiome agents to interact with the microtumors through a synthetic or natural membrane.

Embodiments described herein permit four-dimensional analysis (including three spatial dimensions of the microtumor and time) of a three-dimensional replica of a patient’s tumor, the replica microtumor being derived from a patient biopsy. The dimensions and morphologies of these microreplicates are controlled and pre-defined with hydrogels and chip design. Control parameters are manipulated in order to replicate tumor development in a human/animal body to arrive at a personalized therapy. Microreplicates are standardized; therefore, the process of their growth/development can be highly automated, which increases testing/experiment throughput. Testing and comparing results of various modes of therapy including drugs, chemotherapy, immunotherapy, and radiotherapy, including their combinations, toxicity, etc., can be implemented by embodiments described herein. Microfluidics on the chips enables the capture and analysis of downstream products, such as DNA, RNA, metabolomics, and proteomics. Automation and integration of spectroscopy enables high-performance, multi-modal quantification of tumors, their interaction with therapies in light of external conditions (e.g., gravity). Thus, the embodiments provide predictive, personalized oncology.

Tumor-on-a-chip (TOC) systems disclosed herein grow patient-derived microtumors, tumor fragments, or bio-blobs, as miniature microcopies of patients’ original tumors, ex vivo. It should be understood that the term “chip” can be applied to multi-core designs/implementations, such as plates, cartridges, etc. These TOC systems are able to form and grow the microtumors that are derived from patient biopsies in their native state, preserve the tumor microenvironment, and replicate the tumor behavior so that it can be used to predict the behavior of patient tumors. Disclosed processes involve scalable, inexpensive, and automated tumoroid platforms that can prepare samples within 10 minutes, enabling a wide range of assays in under a week. An advantage of the disclosed device is its ability to test multiple drugs, each at varying doses. This allows the selection of optimal forms of treatment for the patients, hence reducing failure rates. Also, using this invention, the microtumors and/or bio-blobs are placed in pre-defined location, with pre-defined size and shape, that enables quantifying the changes in their shape and size over time.

The preparation steps of chips disclosed herein are straightforward and can be completed by a non-expert user after simple training. Unlike alternative TOC technologies, each of the microtumors grown in the biochips are identical in size and shape, which facilitates statistical analyses. Further, the predefined tumor boundaries enable us to precisely track cellular invasion and monitor variations among samples, automate imaging, and collect high-throughput datasets.

Disclosed chips and processes are used to determine the best course of treatment on an individualized basis. In some implementations, determination is made to stop an ineffective treatment. Results can be provided in only 5 days and do not impact the length of treatment. Thus, disclosed systems can compress a multi-week trial-and-error therapy cycle into a single pre-treatment diagnostic analysis, enabling oncologists to prescribe only the most effective treatment for each patient. Tumoroids are embedded within a secondary hydrogel that resembles the natural pharmacodynamics of the body and feeds the cancer cells with nutrients and chemical cues. A resorbable hydrogel structure allows the tumoroids to grow long-term and invade surrounding tissues if an effective treatment is not applied, similar to their natural behavior inside the body. These multi-gel extracellular structures mimic the pharmacokinetics of drug delivery Disclosed chips and processes culture live patient-derived tumor cells inside a microfluidic platform, exposes them to different therapies, and measures their real-time response before administering a treatment to the patient.

### BRIEF DESCRIPTION OF THE DRAWINGS

FIG. 1 is a perspective view of a first unit of a multi-layer biochip according to one embodiment of the invention.

FIG. 2 is a perspective of a second unit of the biochip of FIG. 1.

FIG. 3 is an exploded view of the layers making up the biochip of FIGS. 1 and 2.

FIG. 4 is a sectional view of an alternative embodiment of a multi-layer biochip, with the first unit being located on the bottom and the second unit on the top of the biochip.

FIG. 5 is a plan view of the biochip of FIG. 4.

FIG. 6 is a plan view of alternative embodiment of the biochip.

FIG. 7 is a see-through perspective view of the biochip of FIG. 5.

FIG. 8 is a schematic of a biochip according to an alternative embodiment of the invention.

FIG. 9 is a flowchart showing a process of using a biochip according to one embodiment of the invention.

FIG. 10 shows a schematic for microfluidic chip used as a precise gradient generator chip to identify patient-specific optimal drug doses, down to minimum effective or resistance thresholds.

FIG. 11 shows the results of a tumoroid being exposed to gradients of dosages of a therapy.

FIG. 12 is an exploded view shows how a microfluidic chip of FIG. 10 may be constructed from multiple layers.

FIG. 13 shows a system comprised of four microfluidic chips of the type shown in FIG. 10.

FIGS. 14-17 show a system using an array of tumoroids in a single large hydrogel chamber, with multiple inlets and a single outlet, wherein the system automatically mixes different treatments and applies them in a variety of ratios, to assess toxicity and synergistic effects of 2 to 5 drug combinations.

FIG. 18 shows an array of tumoroids as may be used in the chamber of the system shown in FIGS. 14-17.

FIG. 19 shows initial and subsequent conditions of a chip monitoring the growth and invasion of a pre-defined tumor with respect to preloaded compartments, indicating how certain materials such as growth factors, drugs, chemicals such as caffeine or tobacco, can activate tumors metastasis or increase cancer aggressiveness. Movements or tilting of the tumor towards each surrounding compartment indicates tendency to invasiveness due to that particular agent.

FIG. 20 is a schematic showing the interface between the cell chamber and a preloaded compartment in a chip device according to a different embodiment of the type shown in FIG. 19.

FIG. 21 shows the response of the tumor to preloaded compartments in a chip according to a different embodiment of the type depicted in FIGS. 19 and 20.

FIGS. 22 and 23 are exploded views of a multilayer biochip having a reservoir and membrane underneath the cell chamber for holding microtumors, allowing a patient’s tumor replica to be exposed to a hypoxic environment, permitting the growth and maintenance of live microbiomes, that can show alternations in treatment responses due to the effect of microbiomes.

FIG. 24 is an exploded view of several layers of the chip shown in FIGS. 22 and 23, in particular layers that define the hypoxic chamber.

FIG. 25 is a plan view of the layer defining the hypoxic chamber of the biochip depicted in FIGS. 22-24.

FIG. 26 shows a representative process for tumor dissociation and biochip presentation.

FIG. 27 shows a representative process for tumor dissociation quality control and tumoroid preparation.

### DESCRIPTION OF ILLUSTRATIVE EMBODIMENTS

Embodiments of the present invention preferably use a series of hydrogels (for example, dopamine-agar, Photocol, Photogel, PEGDa, Agarose-Col, Hyaluronan, Vitrogel. Several natural hydrogels can be used as primary (cell + gel) and surrounding secondary gels. The cells may be mixed with thermoset hydrogels (e.g., solubilized basement membrane preparation (gelatin methacrylamide (GelMA), gelatin, agar, agarose, carrageenan) or light-curable hydrogels (e.g., 4-arm-PEG acrylate). To encourage polymerization between hydrogel types, visible light photoinitiators (e.g., LAP, a photoreactive crosslinker (Photo Initiator Lithium phenyl-2, 4, 6-trimethylbenzoylphosphinate) are used at a concentration of 0.5-20.0% in solution with the secondary hydrogel 50% 4-ARM PEG (4arm PEG Acrylate, MW 20000), and diluted with PBS (Phosphate Buffered Saline pH 7.4 (1X)) before pouring into the biochip cell chambers. Without the dilution, the hydrogel solution may not set correctly, and it will collapse in on itself when the stainless-steel pins are removed after UV curing. The cells encapsulated in hydrogels are delivered to the chambers in a condition to permit continuous growth.

The biochips house the microtumors and facilitates the dynamic flow for biologically relevant tumor response. In some implementations, microtumors are shaped to mimic expected shape within a human body, e.g. elongated along a bone or a blood vessel, spheres, or flattened. The biochips begin as flat sheets of Polycarbonate, Polymethacrylate, Polydimethylsiloxane, or other biocompatible polymer sheets of varying thicknesses, usually 1 to 8 millimeters. Using RDWorksV8, the design of the biochip is separated by its functional layers, consisting of three solid PDMS layers and two double-sided adhesive layers for bonding the solid layers together. Each layer has a defined thickness for optimal function and is precisely cut by the Boss Laser laser-cutter. These biochip pieces are subjected to UV sterilization for a few hours before further handling. The bottom layer and middle layers of the biochip are laminated together using 3M double-sided adhesive.

Once the layers are adhered, the secondary hydrogel, 50% 4-ARM PEG, 2% LAP, and FBS, is poured into the lanes, leaving enough volume for the primary hydrogel. Eighty 1-millimeter stainless steel pins arrayed in five rows in a double-layered PDMS sheet are embedded in the secondary hydrogel to create uniform wells to house the primary hydrogel and tumor spheroids. The assembly is then placed in a 4-degree Celsius fridge and undergoes UV curing via a powerful UV lamp for 10 minutes or even up to 45 minutes, depending on the UV lamp’s wattage. Once the secondary hydrogel is cured, the primary hydrogel cover (PhotoCol Methacrylated Type I Collagen) is combined with the dissociated patient-derived tumor cells using a 1-milliliter syringe.

Due to the small spheroid wells, using a high-gauge needle allows for easy aspiration and dispensation of the hydrogel-tumor cell mixture into the wells the stainless-steel pins created. Nuclear labeling dyes NucBlue^{TM} Live Cell Stain ReadyProbes^{TM} (Life Technologies Corporation, Eugene, OR, USA) and Ethidium Homodimer III (EthD-III, Thermofisher Scientific) are added to the spheroids to help visualize living and dead cells, respectively. The top layer is adhered to the middle layer using the same 3M double-sided adhesive, and then the whole construct is pressed under heat for 2 minutes to laminate all layers together fully. The inlet and outlet ports are sealed with 16mm-by-16mm Poly (methyl methacrylate) (PMMA) squares to discourage leaks at the intersection between biochip and peristaltic pump tubing. Once the chip is sealed, it is placed in the 37-degree Celsius incubator to equilibrate the cells for an hour. During this incubation, the cells form into a spherical tumoroid within the hydrogel. At this time or any point over the next 24 hours, images are taken in z-stacks at 4x or 10x magnification to monitor cell morphology using the Keyence BZ-X810 All-in-One microscope (Keyence, Itasca, IL, USA). The microtumors are evaluated at this point to make sure the following conditions are applicable: proper cell viability (which is specifically important on specimens that has been pre-exposed to radiation, cancer drugs, or have received neo-adjuvant chemotherapies) and proper cell count. More than 500 cells are preferred, with a viability of at least 50% but preferably 75%. The attachment of the microtumors to the surrounding gel should be intact and uniform. The microtumors should not include any large size (50 micrometers or larger) bubble and cells should be relatively uniformly distributed.

The microfluidic tumor-on-a-chip systems described herein are used after a patient is diagnosed with a disease or disorder, particularly cancer (which may be pathogenic or benign). Cells from the tumor biopsy can be obtained from the pathology lab, grown in the system, and screened against multiple drugs in up to two weeks. The results are then conveyed to the primary oncologist allowing them to make a better-informed decision on which drug they decide start the therapy with first. In some implementations, the results evaluated to predict metastasis and set up additional patient monitoring procedures. This personalized approach improves the efficacy of therapy and reduces the time and costs that are associated with treatments.

Patient-derived microtumors can be grown and preserved inside the optimized hydrogels described herein for up to four weeks in vitro. The microtumor represents the real tumor environment at a cellular and molecular level. The scaffolding structure of the microfluidic chips, the multi-hydrogels, and the continuous controlled supply of nutrition and oxygen allow for the cancer cells to grow and function similar to their in vivo performance. The platform described herein allows multiple combinations of potential therapies to be screened allowing the evaluation of optimal combinations for treatment. Various doses can also be tested to determine effective doses for particular patients. This information would allow oncologists to determine more effective treatment strategies.

Described herein are a tumor-on-a-chip systems, which can grow a subject’s tumor biopsy-derived cancer cells outside the body. Tumor spheroids can be formed in the hydrogel environment allowing the growth and viability of the cells to be monitored. With these systems, microtumors can be produces inside the microfluidic chip, creating a platform that can test the efficacy of therapies against a subject’s own cancer cells. These systems offer personalized cancer screening for evaluating the different possible treatments prior to starting therapy.

Growing ex vivo microtumors from a subject’s cancer cells in the system described herein is done within a 2-week period and the device provides a substrate to test the possible treatments. The designs allow the growth of patient-derived cancer cells as spherical microtumors with predefined sizes and shapes. The characteristics of the microtumors can be monitored and the efficacy of the drugs can be determined based on an individualized basis. The devices can be utilized in hospitals, and cancer centers and institutes where it would improve the success rates of therapies, reduce the costs and duration of treatment, and provide a personalized prescription for each patient. Pharmaceutical R&D departments and laboratories that work in the field of cancer can benefit from these devices by using them to evaluate their newly developed drugs and drug delivery systems, so as to improve the accuracy of their results and reduce the costs and time of testing.

Once the spheroids reach an appropriate size and shape, standard National Comprehensive Cancer Network (NCCN)-recommended therapies are added to circulating media in various concentrations determined by the specific study. If the study is retrospective, the therapies employed are the drugs the patient was recommended to undergo by their oncologist. If the study is prospective, the NCCN-recommended therapeutics are used. The therapy-containing media will circulate for about a week while images are taken at critical time points for analysis.

Fluorescence microscopy may be employed with live or dead cell assays, with three dyes used in rotation: NucBlueTM Live Cell Stain ReadyProbesTM (Life Technologies Corporation, Eugene, OR, USA), NucGreenTM Dead 488 (Life Technologies Corporation), and NucRedTM Live 647 (Life Technologies Corporation) along with the cell-impermeant viability indicator, Ethidium Homodimer-1 (EthD-1; Thermofisher Scientific), for red staining. The imaging may be carried out using a Fluorescence Microscope (BZ-X810 All-in-One Fluorescence Microscope; Keyence, Itasca, IL, USA), which is capable of automated z-stack imaging and facilitates in-depth analysis of the spheroids. A traditional ZEISS Axiovert 200M inverted fluorescence microscope (Carl Zeiss AG ZEISS, Oberkochen, Baden-Württemberg, Germany) for phase contrast microscopy and quick imaging of cells during cell culture may also be used, with occasional application in fluorescence microscopy.

Cell counting and sorting are done using the images. An assessment is made of the ratio of living cells to dead cells due to the therapy administered during treatment. If the treatment course lasted a week, images of every tumor in the chip were taken every day. These cell populations change relatively quickly, so images are taken daily to record accurate measurements.

Patient tumor viability may be calculated by dividing the live cell population by the total cell population per spheroid. Depending on the fluorescent image quality and specific dye uptake of the cells, after data corrections based on the colocalization of dyes (dead and alive cells), if applicable. Each spheroid may be imaged every 12 hours in the chip to get a broad picture of how the tumor is being treated by the specific therapeutic of interest. For a single patient’s microtumors, the spheroids can be treated in a replicate of n=5. This ex vivo/in vitro experimental setup allows for rapid determination of therapeutic efficacy based on cell viability.

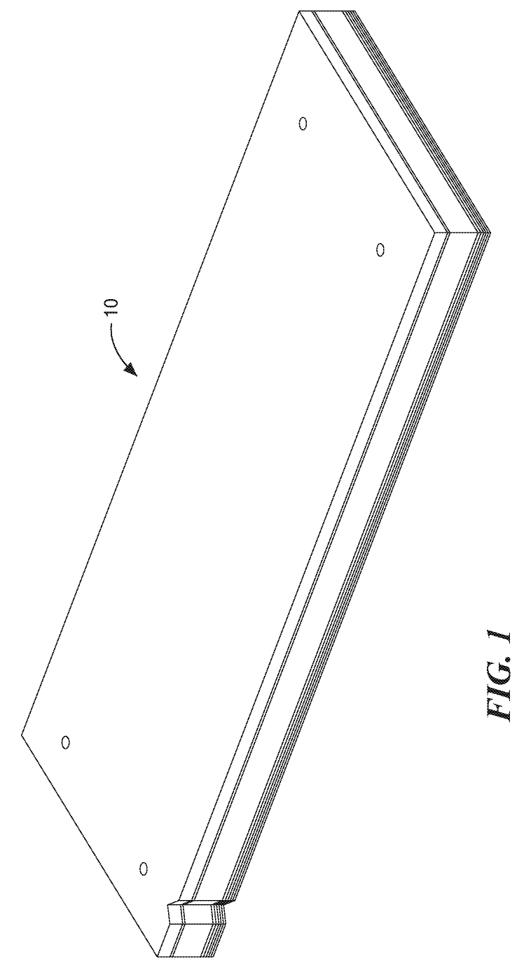

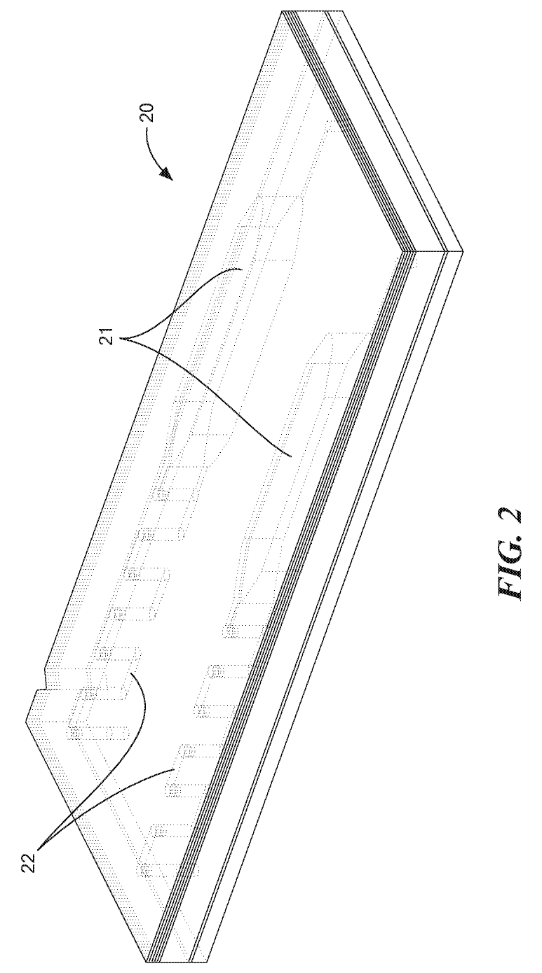

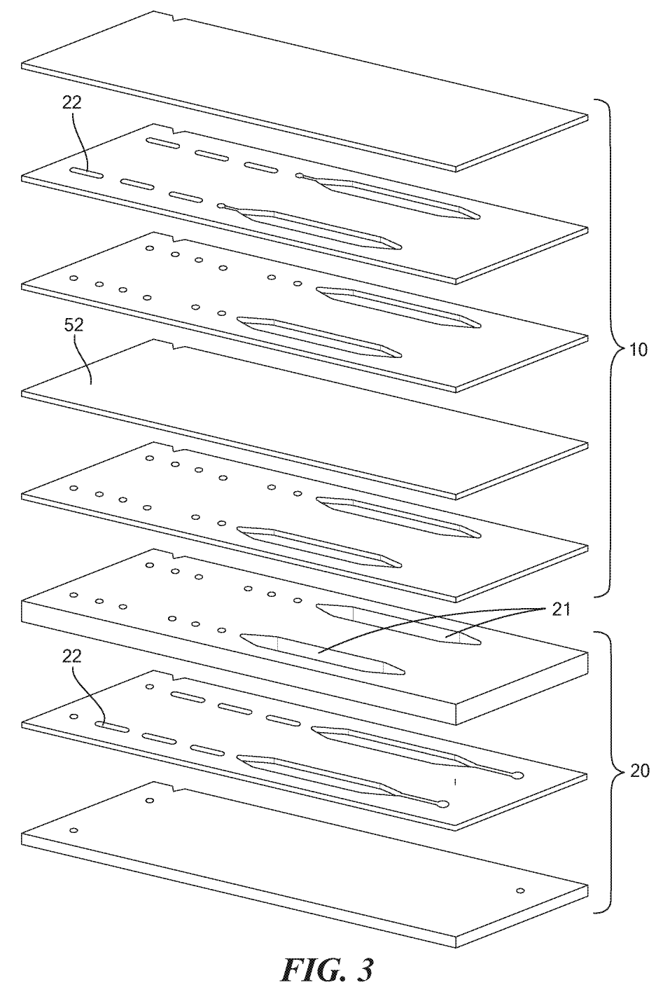

FIGS. 1-3 show a multi-layer biochip according to one embodiment of the invention. The biochip is comprised of two units, a first unit 10 shown in FIG. 1 and a second unit shown in FIG. 2. FIG. 3 shows an exploded view of the layers making up the biochip. Five layers make up the first unit 10, and three layers make up the second unit 20. The second unit 20 defines two hydrogel chambers 21 as shown in FIGS. 2 and 3. Channels 22 are defined in each of the first unit 10 and the second unit 20. The first unit 10 includes a membrane layer 52, which defines the tops of the hydrogel chambers 21 when the first and send units are joined together. In one embodiment, the membranes have different pore sizes, ranging from 200 nm to 2000 nm.

A plurality of hydrogel-encapsulated tumoroids are placed in each hydrogel chamber 21 before the first and second units joined to each other. During the exposure and analysis phases, when the tumoroids are exposed to chemotherapeutic drugs or other compositions and then analyzed, the first unit 10 and the second unit 20 are joined together, preferably in a sealed manner.

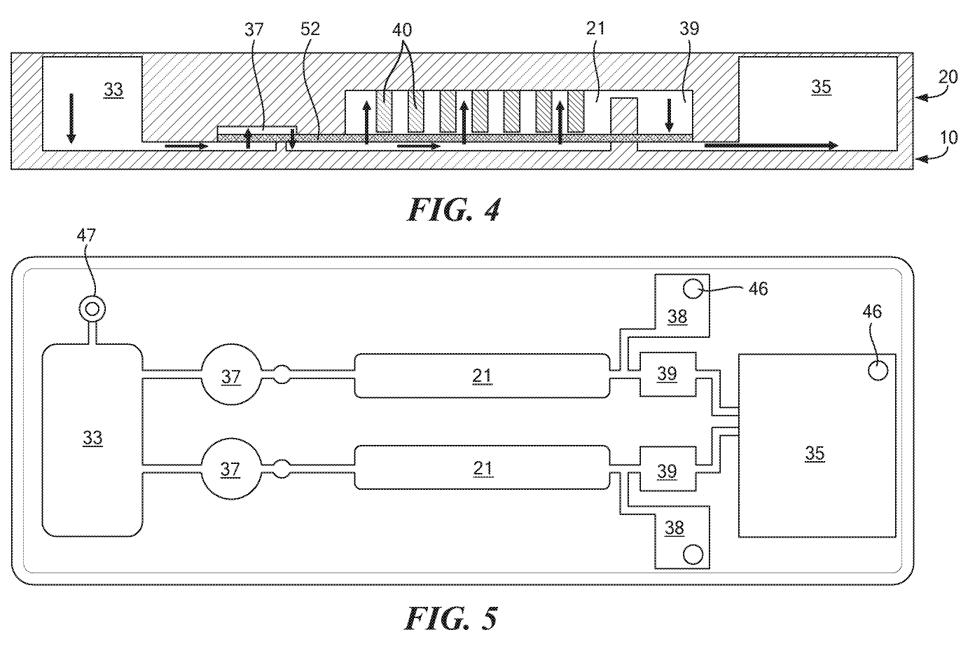

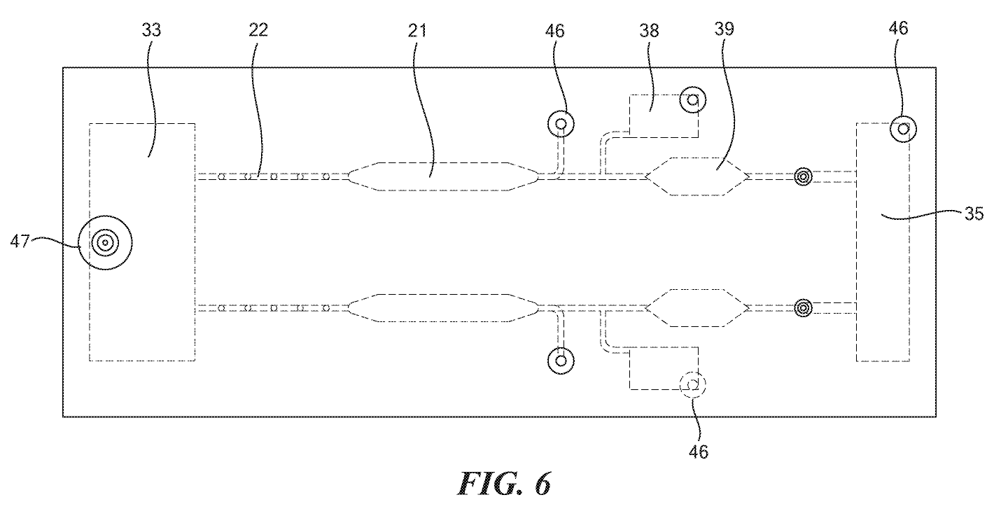

FIGS. 4 and 5 respectively show sectional and plan views of an alternative embodiment of the biochip. The biochip also includes a reservoir 33 for holding a media, such as saline. The reservoir 33 is defined by at least one or both of the first and second units. Note that, unlike the arrangement shown in FIGS. 1-3, the first unit 10 is located on the bottom in the FIG. 4 view.

The reservoir 33 may be filled with an inlet 47. Mixing chambers 37 are also provided in the biochip, each of which may be defined by at least one or both of the first unit 10 and the second unit 20, the mixing chamber being adapted to receive a media and a drug or other composition.

The hydrogel chamber 21 includes a plurality of hydrogel-encapsulated tumoroids 40. The hydrogel chambers 21 may also include a hydrogel with uncured voids for receiving the hydrogel-encapsulated tumoroids 40.

The biochip also includes mixing chambers 37, which are located upstream of the hydrogel chamber 21 and downstream of the reservoir 33. Downstream of the .hydrogel chambers 21 are located cell collection chambers 37, which collect cells that are shed by the tumoroids. Also downstream of the hydrogel chambers 21 are media collection chambers 38 for collecting media flowing from the hydrogel chambers 21. A terminal collection chamber 35 may be located downstream of the cell collection chambers 39. Vents 46 may be provided in the media collection chambers 38 and the terminal collection chamber 35 to allow gas to escape from the microchip.

A single membrane 52 layer may be used to filter the media entering into the mixing chamber 37, filter the drug-media mixture entering into the hydrogel chamber 21, and filtering out the shed cells to prevent them from flowing out of the cell collection chambers 39. Alternatively, separate membranes may be used at the different locations.

The membrane 52 may be a layer in the first unit 10. When the first unit 10 and second unit 20 are joined together, the membrane 52 defines the hydrogel chamber 21. The membrane 52 permits the flow of a mixture of the media and the drug into the hydrogel chamber 21, but holds the hydrogel-encapsulated tumoroids 40 in the hydrogel chamber 21.

The biochip is preferably designed with transparent windows allowing the tumoroids in the hydrogel chambers 21 to be imaged and analyzed as the tumoroids change in size and shape and as cells migrate during the analysis process. Windows may also permit imaging and analysis of shed cells collected in the cell collection chambers 39.

In addition to image analysis of the tumoroids and cells, testing for biomarkers may be performed by providing needle access ports to the hydrogel chambers 21 and the cell collection chambers 39, through which ports cells may be collected and analyzed outside of the biochip.

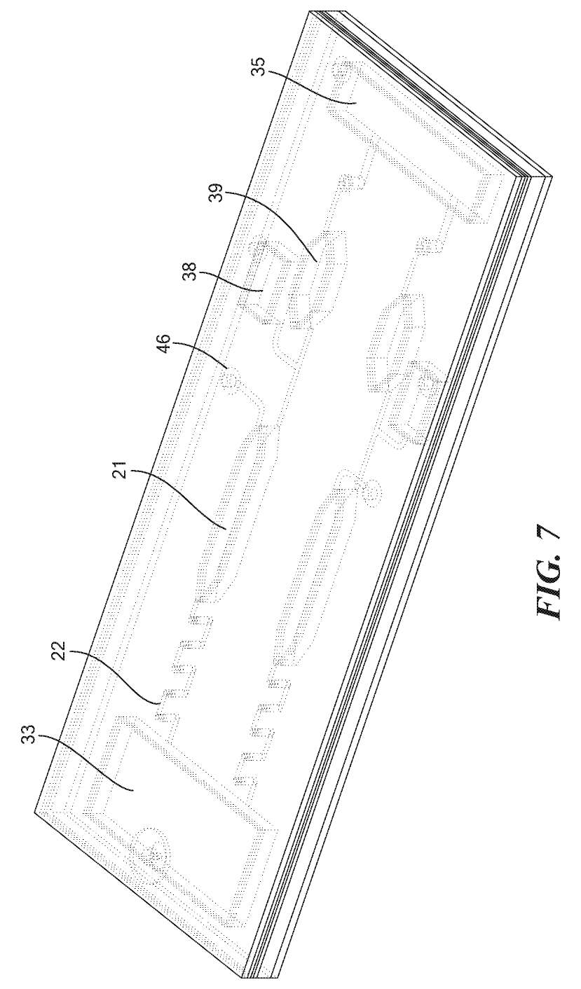

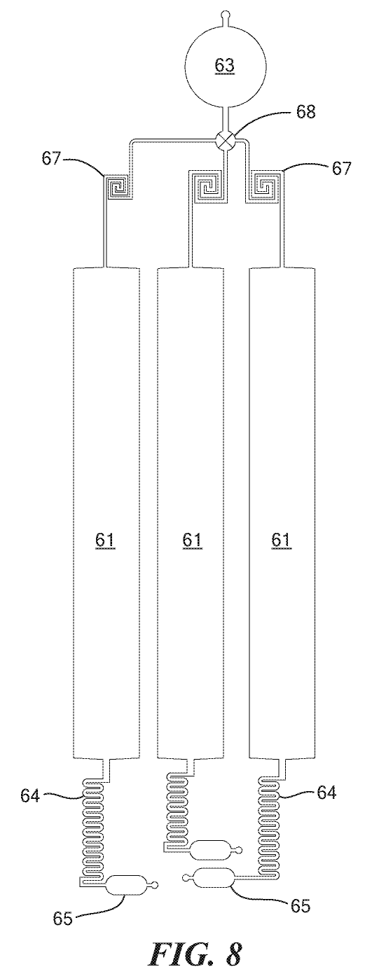

FIGS. 6 and 7 are respectively plan and see-through perspective views of an alternative embodiment of the biochip. This embodiment includes a reservoir 33 for holding media and an inlet 47 to the reservoir 33. Channels 22 connect the reservoir 33 to the hydrogel chambers 21. The channels 22 themselves may act as mixing chamber, wherein a drug or other composition may be mixed with the media from the reservoir 33, and the mixture may then flow downstream to the hydrogel chambers 21.

A plurality of hydrogel-encapsulated tumoroids may be placed within each hydrogel chamber 21. Each hydrogel chamber 21 preferably includes a cured hydrogel providing a plurality of uncured spaces, each of the uncured spaces being capable of being filled with a hydrogel-encapsulated tumoroid. The hydrogel defining the uncured spaces may be cured by a different mechanism from the hydrogel encapsulating the tumoroid.

Downstream of the hydrogel chambers 21 are cell collection chambers 39 and media collection chambers 39. Downstream of the cell collection chambers 39 is a terminal collection chamber 35. Vents 46 are provided in various locations on the biochip to vent gas.

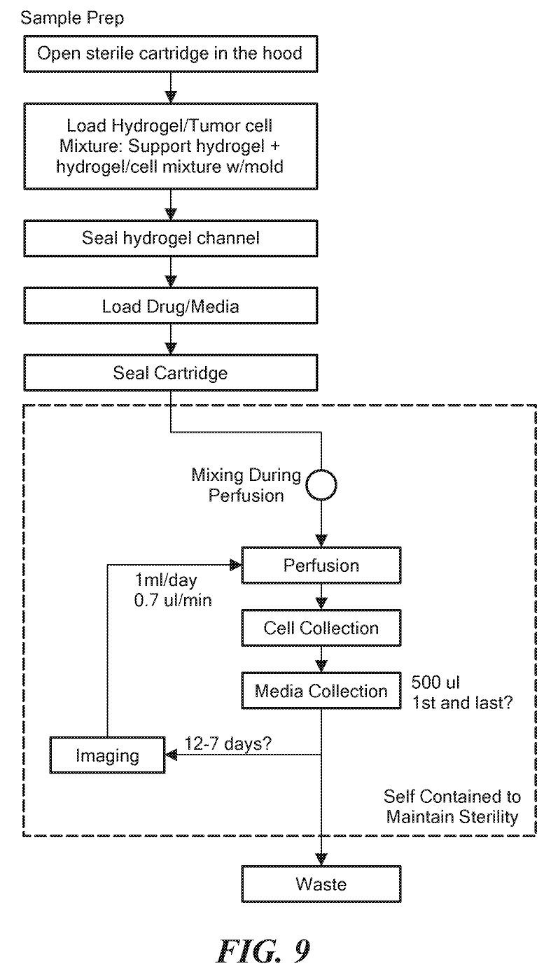

FIG. 8 is a schematic of a biochip according to an alternative embodiment of the invention. A reservoir 63 holds the media. A valve 68 controls the flow of media through three different flow paths. Each flow path includes a mixing chamber 67, a hydrogel chamber 61, a flow resistor 64, and a collection chamber 65.

In alternative embodiments, the biochip is provided with first follow-on collection chamber downstream of the cell collection chamber. A second follow-on collection chamber may also be provided downstream of the first follow-on collection chamber. The cell collection chamber is adapted to hold cells shed from the tumoroid within about the previous day. The first follow-on collection chamber is adapted to hold cells shed from the tumoroid from about two to five days earlier. The second follow-on collection chamber is adapted to hold cells shed from the tumoroid from more than about five days earlier. Each of the chambers in this series may be provided with windows to permit the imaging of the shed cells contained therein. In a biochip with a terminal collection chamber, a window may also be provided for a terminal collection chamber so that shed cells may be imaged over a period of time. Some or all of the chambers may be provided with a needle-access port to permit the removal of the shed cells from the sealed biochip for analysis outside of the biochip.

A preferred method for monitoring cell growth and viability includes providing one of the biochips described above; isolating cells from a subject or a human or animal cell line; encapsulating the cells in the second curable hydrogel; inserting the hydrogel-encapsulated cells into at least one of the uncured spaces while the hydrogel chamber while the first and second units are not joined to each other; joining the first and second units together; providing a media; providing a drug; mixing the media and the drug; causing the mixture of the drug and media to flow into the hydrogel chamber (preferably through a filtering membrane), and analyzing the hydrogel-encapsulated cells in the microfluidic device.

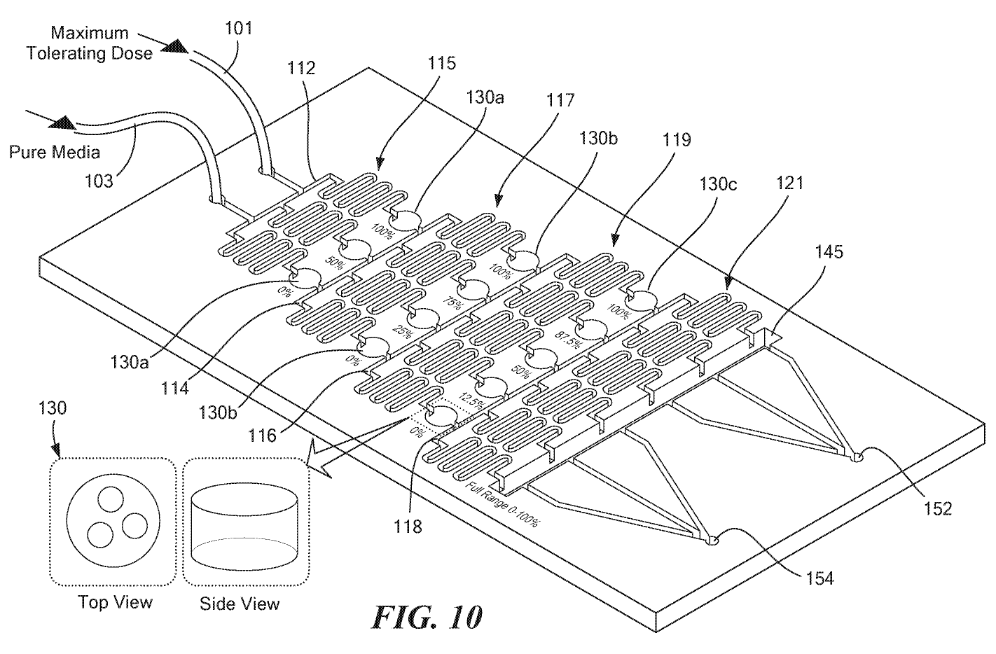

FIG. 9 is a flowchart showing a process of using a biochip according to one embodiment of the invention.

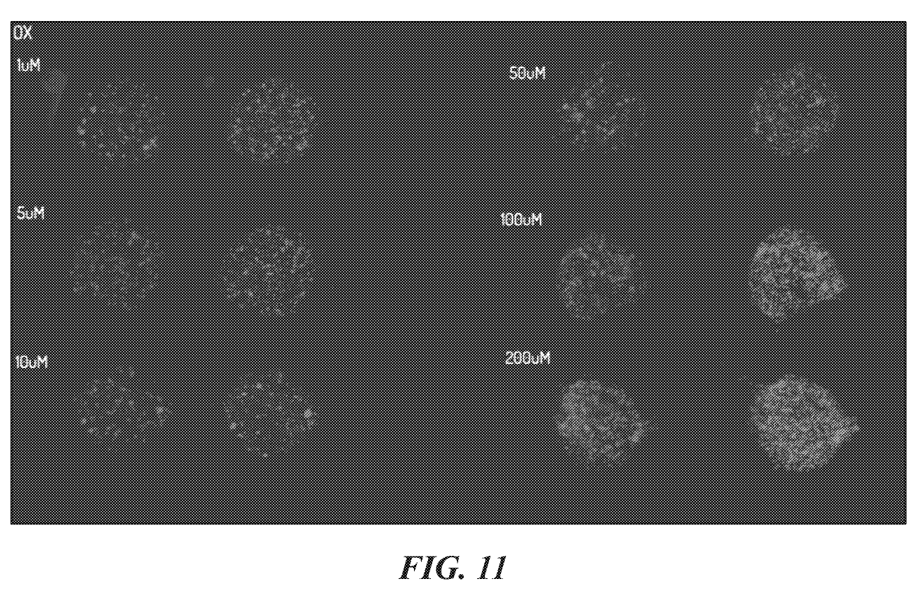

A microfluidic chip for generating a precise gradient to identify patient-specific optimal drug doses is shown in FIG. 10. In a preferred embodiment, a first inlet 101 of the chip shown in FIG. 10 receives a maximum safe total dose of a medication, such as a chemotherapy drug or the like, carried in a media (such a saline). The second inlet 103 receives just pure media (such as saline) By mixing a maximum safe dosage from one inlet 101 with pure media from the other inlet 103, a full range of different concentrations may be tested on microreplicates from the patient’s tumor. Alternatively, the first and second inlets may respectively provide first and second drugs. In this embodiment, the effect of different drugs ratios on the patient’s tumor may be compared to determine an optimized drug cocktail composition. The microreplicates are placed in the various wells 130a-c in the chip in order to be exposed to the different concentrations of the medication.

The first inlet 101 and second inlet 103 lead to an inlet-connecting channel 112 fluidly connecting the first and second inlets. The first and second inlets connect to the inlet-connecting channel 112 at different points. This inlet-connecting channel 112 in turn leads to three flow paths having flow resistors 115 as shown. These flow paths connect to the inlet-connecting channel 112 at three different points. Depending on the relative pressures, the top flow path will have a high (preferably 100%) concentration of the drug. The bottom flow path will have a minimum (preferably 0%) concentration of the drug, while the middle flow path will have a concentration of about 50%. Each of these three flow paths lead to a separate well 130. Each of these well 130 contains a microreplicate of the patient’s tumor.

Moving further to the lower right in the chip depicted in FIG. 10, each of these wells lead to a first intermediate channel 114 which connects the first set of wells 130 to flour separate flow paths, each having a flow resistor 117. These four flow paths and the first three wells connect to the first intermediate flow channel 114 at seven different points. The different concentrations flow from the first set of three wells 130a, through the first intermediate channel 114, into the four parallel fluid paths 117, to a set of four wells 130b. Microreplicates may also be included in these wells. Each of these wells 130b receive a different concentration of the drug: preferably, from top to bottom, about 100%, 75%, 25% and 0%.

These four wells 130b in turn are connected at four separate points to a second intermediate channel 116, which in turn is connected at five separate points to five flow paths with flow resistors 119.

Flowing out of these five flow paths 119 are preferably drug concentrations of about 100%, 87.5%, 50%, 12.5% and 0% from top to bottom. Each of these five flow paths 119 flows into a separate well 130c. Microreplicates may also be included in these wells 130c. The different drug concentrations from these five wells in turn flow into an outlet-connecting channel 118 at five separate points.

From six different points, six flow paths with flow resistors 121 extend from the outlet-connecting path 118 flow out of these flow paths. These six flow paths 121 lead to a longitudinal well 145, which may contain a plurality of microreplicates. With this embodiment, precise gradients of concentrations can be produced to identify patient-specific optimal drug doses, down to minimum effective or resistance thresholds.

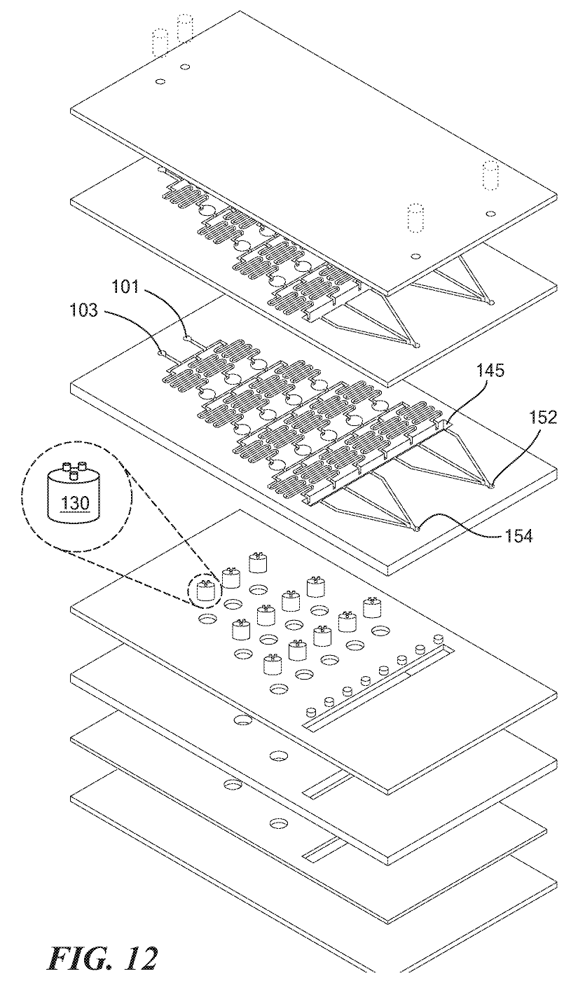

In FIG. 11, test results are shown of different microreplicates being exposed to different concentrations of a medication in a microfluidic device like that shown in FIG. 10. Thus, the reaction of a patient’s tumor to a gradient of dosages can be ascertained.

As shown in the depicted embodiment, the longitudinal may have six outlets. Of these, the three outlets with the higher concentrations may be combined into a single outlet 152, and the three outlets with the lower concentration may be combined into a separate single outlet 154.

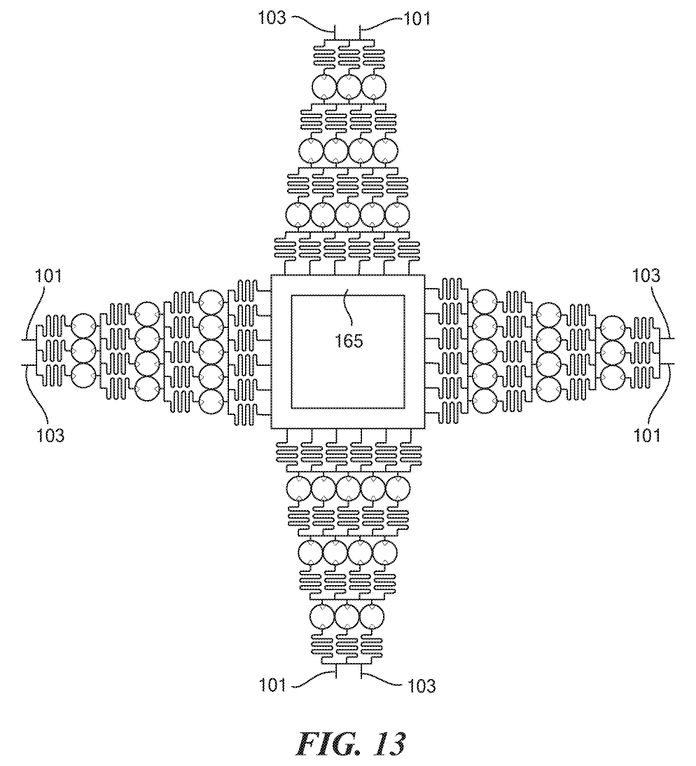

FIG. 12 shows how the microfluidic chip schematically shown in FIG. 10 may be constructed from multiple layers.

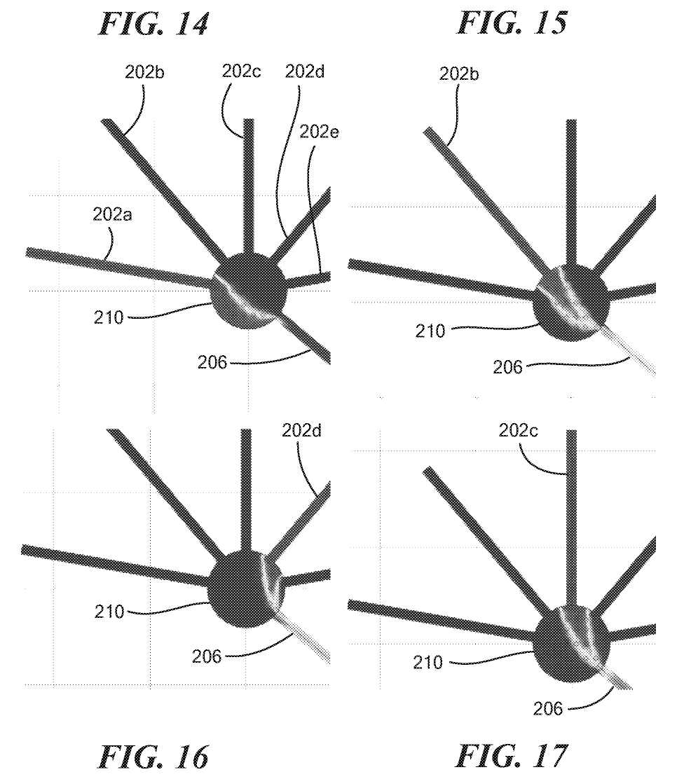

FIG. 13 shows a preferred embodiment, wherein four chips, each of which includes a first inlet 101 and a second inlet 103 like the FIG. 10 embodiment, are connected by a common square-shaped chamber 165, into which microreplicates may be placed. Alternatively, the square-shaped chamber 165 may simply collect waste from the chips. Excess waste may be emptied from the square-shaped chamber 165 during the procedure. Like the FIG. 10 embodiment, microreplicates based on a biopsy from a patient are placed in the various cells, where the microreplicates may be exposed to different concentrations of a medication or multiple medications. For example, each of the four microfluidic chips may be provided with a different drug, and each microfluidic chip may provide a variety of concentrations of its respective drug to the cell chamber.

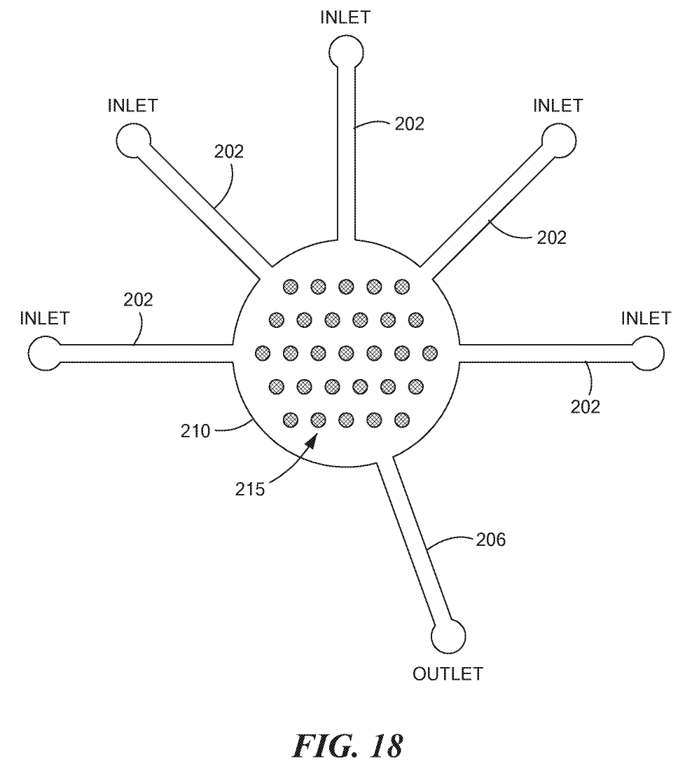

FIGS. 14-17 schematically show a microfluidic device using five inlets 202a-e for introducing different drugs into a cell chamber 210. The device is used to assess the toxicity and synergistic effects of two to five drug combinations The cell chamber 210 may hold an array of hydrogel-encapsulated tumoroids. Each inlet channel 202a-e provides a different medication. A single outlet 206 from the chamber is provided. The cell chamber 210 may also contain a second hydrogel having uncured spaces, into which the hydrogel-encapsulated tumoroids are placed.

Each tumoroid in the cell chamber 210 is exposed to a different ratio of medications based on their positions within the chamber 210. For example, the tumoroids in the lower left of the cell chamber 210 are exposed to more of the medication flowing from the first inlet 202a than to the medication flowing from the fourth inlet 202d. Also, the tumoroids in the upper left of the cell chamber 210 are exposed to more of the medication flowing from the second inlet 202b than to the medication flowing from the fourth inlet 202d. The tumoroids in the lower right of the chamber 210 near the outlet 206 are the most exposed to all of the medications. Based on the different ratios to which each tumoroid is exposed, the system assesses the toxicity and synergistic effects of the different drug combinations.

The shading shown in FIGS. 14-17 indicates the dose level based on mass transfer simulations. With this method, a single chip enables the assessment of multiple combinations and ratios depending on what flow rate is selected for each inlet. With this embodiment, different treatments can be mixed and applied in a variety of ratios, to assess toxicity synergistic effects of two to five drug combinations.

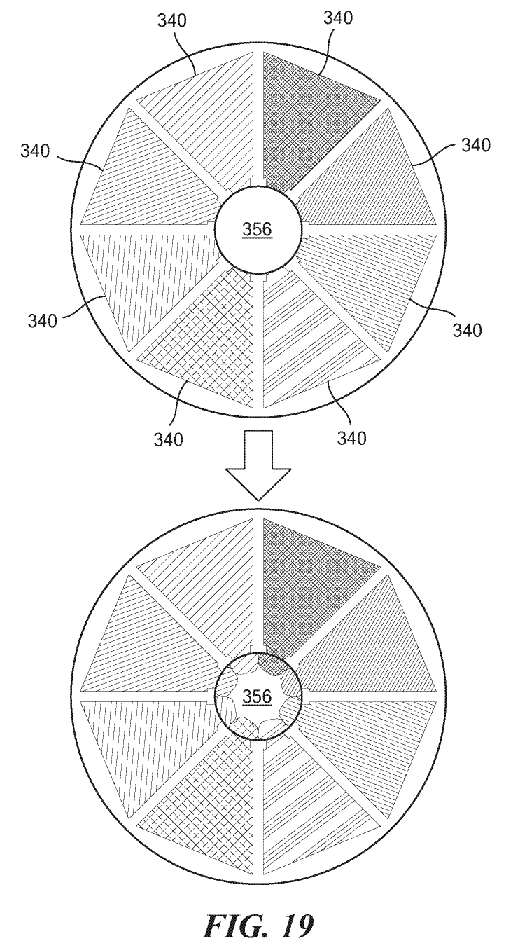

FIG. 18 shows an alternative embodiment with six inlets 202 and a single outlet 206. An array 215 of tumoroids is placed in the single cell chamber 210. Each tumoroid is preferably encapsulated in a hydrogel.

Like the device shown in FIGS. 14-17, the device shown in FIG. 18 is used for evaluating therapies for a tumor. An array of tumoroids 215 is placed in the cell chamber 210 and exposed to different combinations of medication flowing from the inlet channels 202. The method also includes monitoring the reaction of the array of tumoroids 215 to the different combinations of medications. The flow coming from each inlet 202 moves among the tumoroids in the cell chamber 210, ensuring a pre-defined concentration exposure to each of the tumoroids in the array.

The devices shown in FIGS. 14-17 and 18 are preferably made of multiple layers, including an upper layer, a lower layer, and a middle layer. When these layers are combined, the cell chamber for holding an array of tumoroids is formed. As noted above, these tumoroids preferably are encapsulated in hydrogels. The inlet channels and an outlet channel are also preferably defined by the layers.

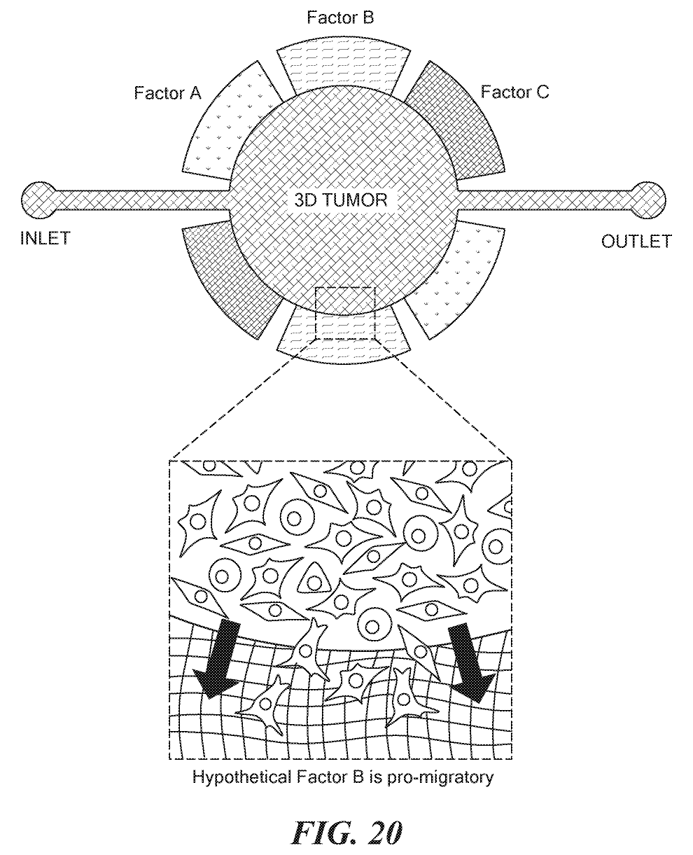

FIGS. 19-21 show systems for evaluating the effect of different compositions on a tumor. In FIG. 19, a microfluidic chip is shown with compartments 340 or reservoirs that are preloaded with different compositions, first in an initial state (top) and after the compositions have migrated into the central chamber 356 (bottom). The central chamber 356 is in fluid communication with each of the pre-loaded compartments 340. A pre-defined tumor is placed into the central chamber 356, such that the tumor substantially fills the central cell chamber 356 and is adjacent each of the compartments 340. The compositions are allowed to interact with the tumor. The tumor’s growth and reaction to the compositions in the preloaded reservoirs or compartments are monitored. The compositions may include a medicament, a potential carcinogen, or combinations thereof. This system can show how certain materials, such as growth factors, drugs, chemicals such as caffeine, or tobacco, can activate tumors metastasis or increase cancer aggressiveness. Movements or tilting of the microfluidic chip urge the tumor towards or away from different compartments and can be used to indicate the tumor’s tendency to invasiveness due to that particular composition.

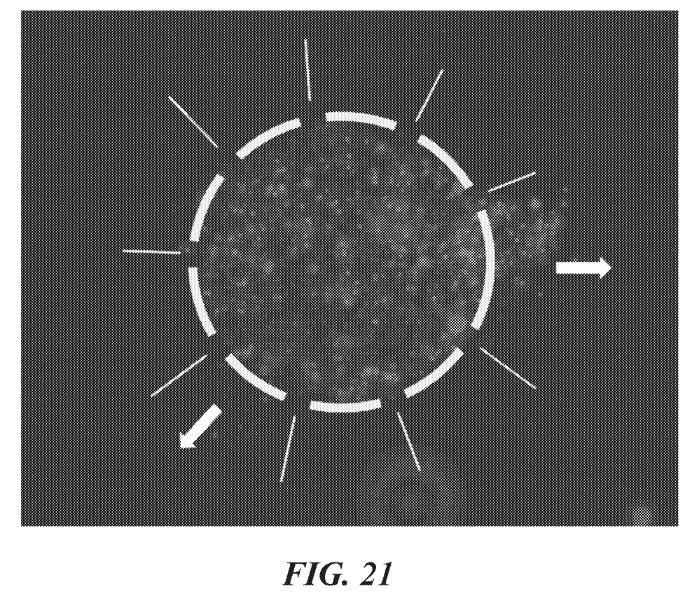

FIG. 20 is a schematic of an alternative embodiment and shows the interface between the cell chamber and a preloaded compartment in the chip. Each surrounding compartment can include hydrogels loaded with an agent (a drug, toxin, growth factor, etc). Alternatively, the surrounding compartments may include the agents in liquid. If the chamber is filled with liquid holding an agent, a membrane is used to ensure separation, while allowing mass transfer and agent penetration from the surrounding chambers, or cell penetration from the central tumor to the surrounding chambers.

The embodiment in FIG. 20 includes an inlet and outlet optional, which may be used for providing nutrients to the tumoroid. Such an inlet and outlet are not required for all embodiments.

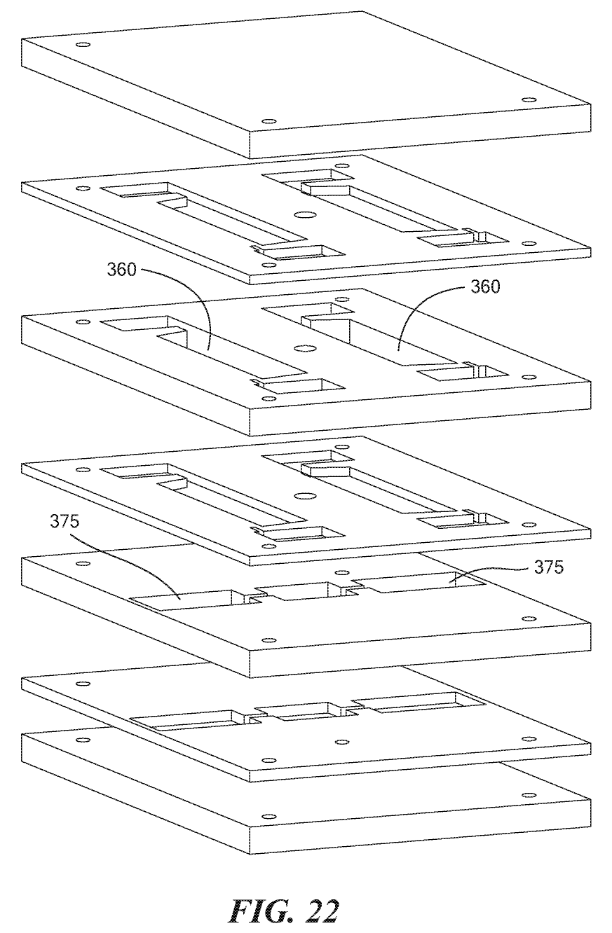

FIG. 21 shows the response of the tumor to preloaded compartments in a chip similar to those depicted in FIGS. 19 and 20, but with a different number of preloaded compartments. The FIG. 19 device has eight compartments, the FIG. 20 has six compartments, and the FIG. 21 device has nine compartments.

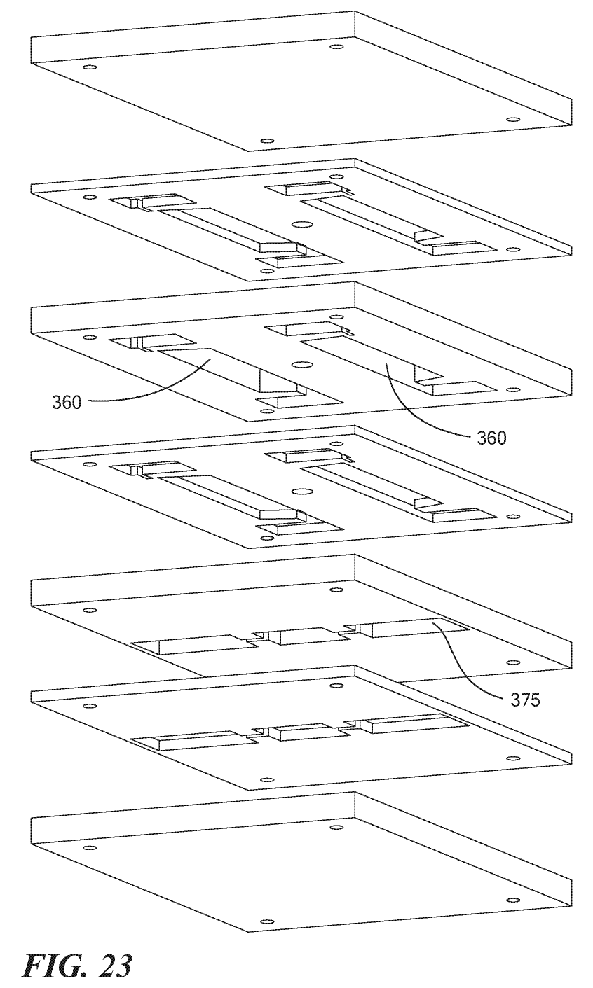

FIGS. 22 and 23 are exploded views of a multilayer biochip having a reservoir and membrane underneath the cell chamber for holding microtumors, allowing a patient’s tumor replica to be exposed to a hypoxic environment, permitting the growth and maintenance of live microbiomes, that can show alternations in treatment responses due to the effect of microbiomes.

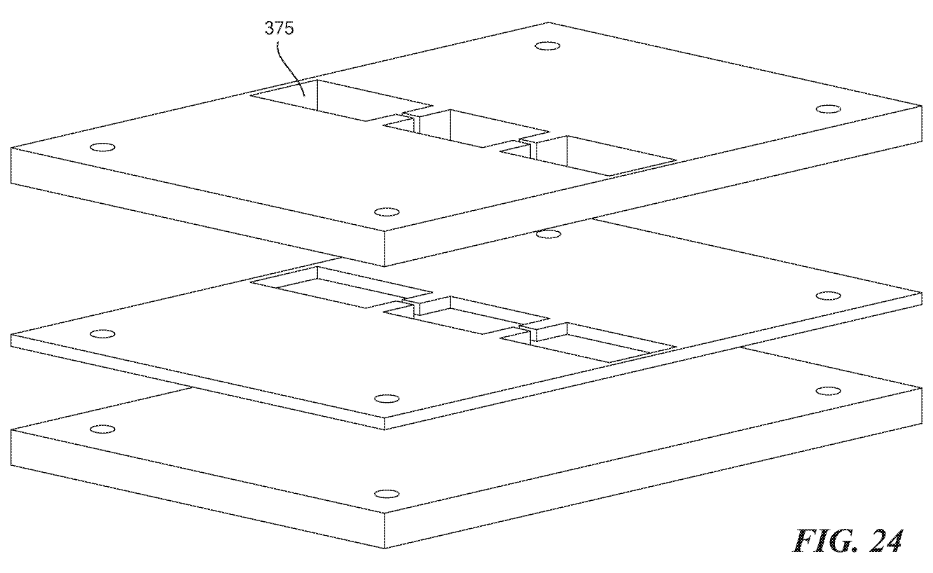

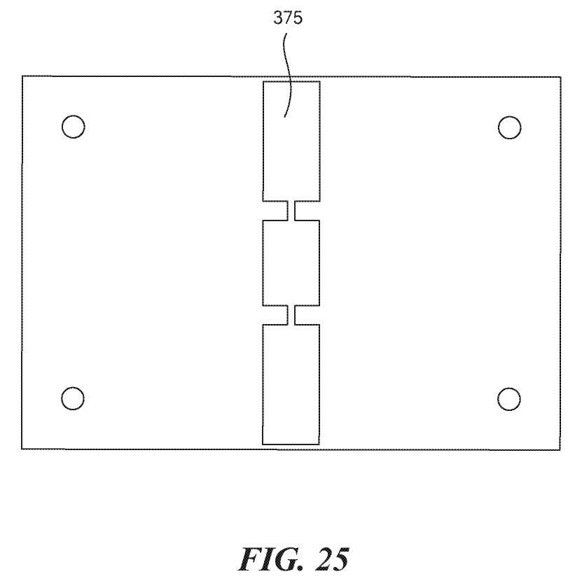

As shown in FIGS. 22 and 23, the biochip may be formed from seven layers, which are assembled together to form the biochip.. Several layers define two separate cell chambers, which preferably hold several hydrogel-encapsulated tumoroids. Several other layers define the hypoxic chamber 375, which may be broken up into three sub-chambers as shown in the figures. FIG. 24 shows three layers defining the hypoxic chamber 375. FIG. 25 is a plan view of a layer defining the hypoxic chamber 375 depicted in FIGS. 22-24.

The hypoxic chamber 375 of the biochip shown in FIGS. 22 and 23 may be separated from the cell chambers 360 by a membrane underneath the cell chamber holding the microtumors. The system allow a patient’s tumor replica to be exposed to a hypoxic environment while growing and maintaining a live microbiome in the cell chamber. The multi-layer biochip may include a plurality of inlets, outlets, and channels in fluid communication with each other. A first middle layer or layers defining the cell-growing chamber is preferably in fluid communication with a culture medium channel. The cell-growing chamber 360 is adapted to hold a population of tumor cells encapsulated in a cured hydrogel. The multi-layer biochip also includes one or more middle layers defining the hypoxic chamber 375 positioned underneath the cell-growing chambers 360, such that the encapsulated tumor cells are exposed to a hypoxic environment.

In a preferred embodiment, the cell-growing chamber further comprises a second cured hydrogel, wherein the second cured hydrogel provides a plurality of uncured spaces, the uncured space containing the cured hydrogel encapsulating the tumor cells. A method using this biochip evaluates the efficacy of a treatment on a subject’s tumor. Tumor cells are isolated from a subject’s tumor, then encapsulated in a curable hydrogel The cells are inserted in the multi-layer biochip. The encapsulated tumor cells are then incubated in the microfluidic device under conditions favorable for tumor cell growth for a period of time. The encapsulated tumor cells are then exposed to one or more therapies for a period of time. Changes and viability in the encapsulated tumor cells are analyzed after the period of time. Accordingly, the efficacy of the therapy on the encapsulated tumor cells is then evaluated. In view of this evaluation, a treatment and/or dosage is initiated, maintained, or modified for the subject from whom the tumor cells were isolated.

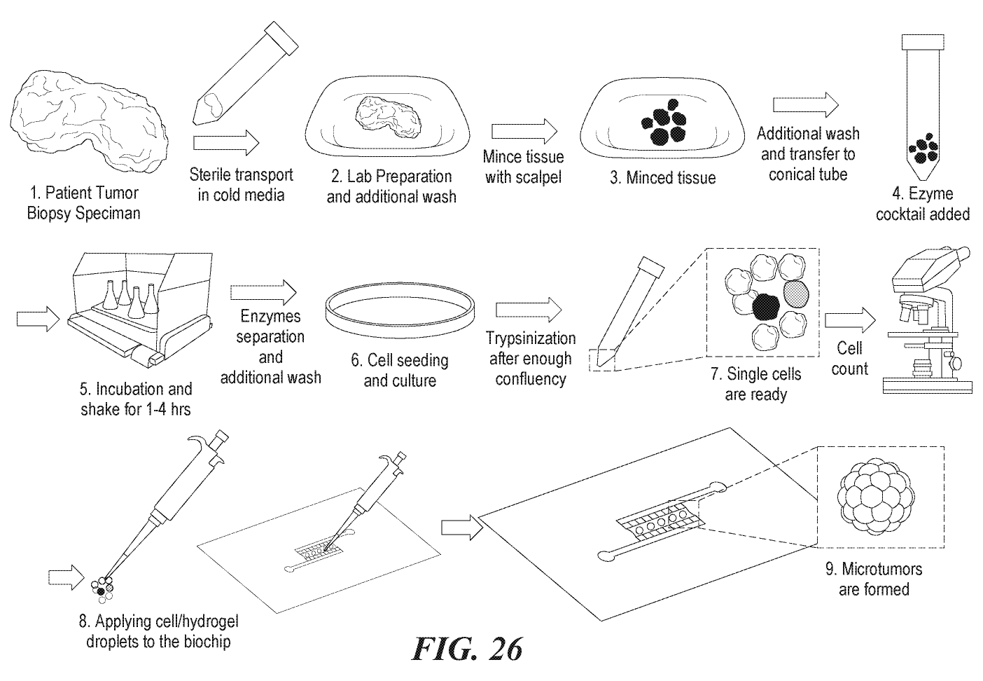

FIG. 26 shows a representative process for tumor dissociation and biochip presentation. Heterogeneous single cell suspensions are obtained from a patient’s tumor needle biopsy samples. Utilizing tumor tissue samples obtained during clinically indicated surgeries and/or biopsies early in the diagnosis process provides sufficient cells to perform the procedures described herein. These tissue samples can be collected via current standard needle biopsy procedure or surgical samples, fresh or viably frozen and stored for a course of tens of years properly. Tissue samples may be sent to a laboratory overnight (preserved in ice or dry ice, for example). (In frozen cases, the samples may be centrifuged to separate from the toxic cryopreservation agents.) The tissues then undergo a series of tissue dissociation steps as shown in FIG. 26.

The dissociation buffer may be prepared by adding Collagenase D (1 mg/mL) and DNase I (100 U/mL) to RPMI-1640 medium supplemented with 10% FBS and 1% Pen-Strep. The buffer may be pre-warmed to 37°C. Colorectal needle biopsy samples are collected using sterile techniques and immediately placed in a tube containing cold RPMI-1640 medium. Upon arrival at the laboratory, biopsy samples are transferred into a sterile petri dish containing 5 mL of pre-warmed dissociation buffer. Using sterile forceps and a scalpel, the biopsy samples are minced into small pieces (approximately 1 mm³). The minced tissue is transferred into a tube containing 5 mL of dissociation buffer. The tube was tightly closed and attached to a dissociator. After the dissociation program, the tube is incubated at 37°C for an additional 45 minutes with gentle agitation. The dissociated cell suspension is filtered through a 40 µm cell strainer into a 50 mL conical tube to remove undigested tissue and clumps. The tube and the cell strainer are washed with 5 mL of RPMI-1640 medium to recover any remaining cells, and this wash is added to the filtered cell suspension. The filtered cell suspension is centrifuged at 300 × g for 10 minutes at room temperature. The supernatant is carefully aspirated without disturbing the cell pellet. The cell pellet is resuspended in an appropriate volume of RPMI-1640 medium supplemented with 10% FBS and 1% Pen-Strep. Cells are counted using a hemocytometer or an automated cell counter, and viability was assessed using trypan blue exclusion. The cell concentration is adjusted to the desired density for downstream applications. Cells are seeded into appropriate culture vessels (e.g., 96-well plates, flasks) and incubated at 37°C with 5% CO₂. All procedures are performed under sterile conditions to prevent contamination. Optimization of the dissociation program and incubation times may be necessary depending on the specific characteristics of the biopsy samples. Additional cell purification steps, such as red blood cell lysis or gradient centrifugation, may be required for specific downstream applications.

If the number of cells is less than minimum requirement for performing the experiment (less than 100 per sample group), the primary cells may be grown in tissue culture flasks, or petri dishes until they reach confluency. They are passaged into T-25 and T-75 flasks and cultured with a DMEM high glucose medium (4.5 g/L), L-Glutamine & Sodium Pyruvate supplemented with 10% FBS (Fetal Bovine Serum, Qualified One Shot), 1% L-glutamine and 1% penicillin/streptomycin at 37 ◦C with 5% CO2. Subsequently, the cells are passaged again upon confluency and remain in the incubator until required for further studies using biochips and 96-well cell culture plate assays or immediately utilized for in vitro testing. Since these cells are not from established cell lines, their growth rate varies from patient to patient, typically taking about a week to reach confluency.

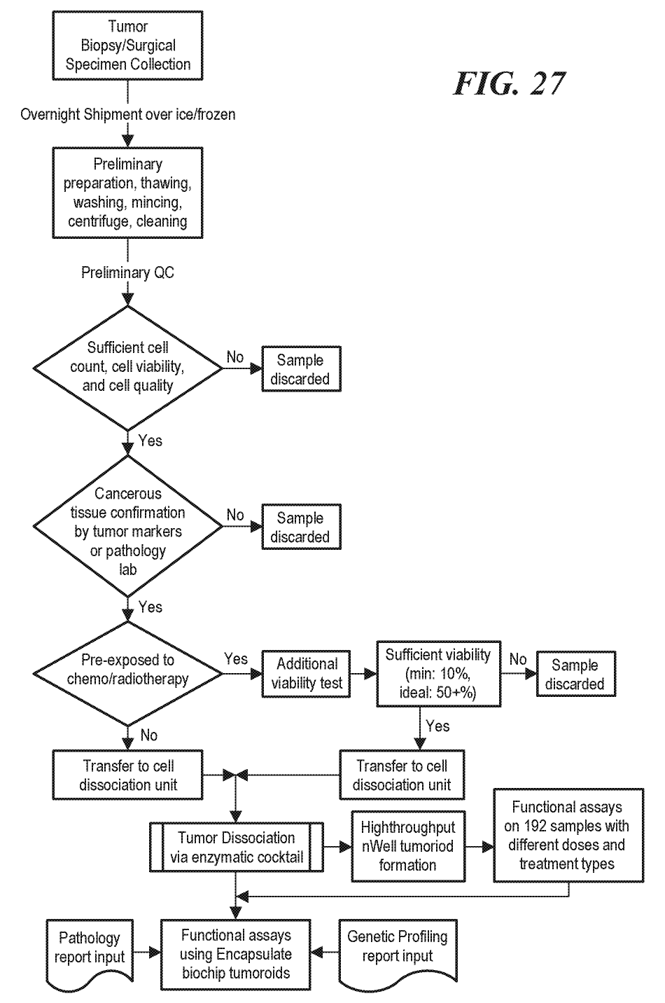

FIG. 27 shows a representative process for tumor dissociation quality control and bio-blob preparation. Cells are trypsinized and lifted to mix with hydrogels and seed in biochips.

In certain embodiments, a microtumor construct is provided that includes a first cured hydrogel forming a continuous scaffold. The scaffold defines a plurality of discrete voids, each void having a predefined geometry and volume. Disposed within each of the voids is a second cured hydrogel that encapsulates a population of viable tumor cells. The interface between the first cured hydrogel and the second cured hydrogel defines a distinct boundary for each resulting microtumor. By virtue of the predefined void geometries and controlled filling process, the plurality of microtumors formed within the scaffold have substantially uniform size and shape across the construct. Such uniformity enhances reproducibility in downstream analyses, including drug response testing and comparative biological studies.

The voids formed in the first cured hydrogel may be cylindrical, spherical, cubic, elongated, or any other suitable geometry, including combinations thereof. In some implementations, the void geometry is selected to approximate physiologically relevant tumor architectures or to enable standardized volumetric measurements. The predefined volume of each void enables precise control over initial tumor cell number and microtumor dimensions.

The tumor cells encapsulated within the second cured hydrogel may be derived from a biopsy or resected tumor sample obtained from a human subject. The cells are preferably patient-specific and may include primary tumor cells, tumor stem cells, or mixed tumor populations. The use of patient-derived cells allows the microtumor construct to serve as a personalized ex vivo tumor model.

In certain embodiments, the first cured hydrogel comprises a collagen-based hydrogel, while the second cured hydrogel comprises a polyethylene glycol (PEG)-based hydrogel. The first hydrogel may provide a structural and extracellular matrix–mimetic environment, while the second hydrogel provides a tunable encapsulation matrix for the tumor cells. In some configurations, the second cured hydrogel is degradable, thereby permitting tumor cell invasion into the surrounding first hydrogel over time. This arrangement enables modeling of tumor invasion dynamics and cell–matrix interactions under controlled conditions.

The first and second hydrogels may be selected and formulated to provide predetermined diffusion characteristics for one or more therapeutic agents. For example, crosslink density, polymer composition, and degradation kinetics may be tuned to regulate diffusion rates of chemotherapeutic drugs, targeted agents, biologics, or other test compounds through the microtumor construct. Such control allows simulation of physiologically relevant drug gradients and exposure profiles.

In preferred embodiments, the microtumor construct is configured for placement within a microfluidic device that delivers one or more test treatments to the microtumors. The microfluidic device may include channels for controlled perfusion of media and drugs, thereby enabling precise spatial and temporal control over treatment conditions. As noted above, in certain implementations, the microfluidic device further comprises a hypoxic chamber positioned adjacent to the microtumor construct and configured to expose the microtumors to a reduced-oxygen environment. This configuration enables modeling of tumor hypoxia and its effects on drug response.

In additional embodiments, a method is provided for generating a treatment recommendation for a subject having a tumor. The method includes obtaining a tissue sample comprising tumor cells from the subject and dissociating the tissue sample to form a cell suspension comprising viable tumor cells. In some implementations, the cell suspension is evaluated to confirm that it contains at least a minimum number of viable tumor cells and meets a predefined viability threshold before further processing.

The viable tumor cells are encapsulated in a curable hydrogel to form a tumor cell–hydrogel mixture. This mixture is introduced into a plurality of predefined voids of a preformed cured hydrogel scaffold to generate a plurality of microtumors. Each microtumor is bounded by an interface between the curable hydrogel and the preformed cured hydrogel. Following introduction into the voids, the curable hydrogel is cured to produce standardized microtumors having substantially uniform size and shape. Standardization of microtumor dimensions enables reliable comparison of treatment responses across multiple conditions.

The standardized microtumors are then placed in a microfluidic device configured to deliver a plurality of test treatments. The test treatments may include one or more drugs administered at different concentrations and/or in different combinations. In some embodiments, the drugs are chemotherapeutic agents selected in accordance with clinical guidelines relevant to the subject’s tumor type. The microfluidic device may be configured to expose different subsets of microtumors to distinct treatment regimens, including multiple concentrations and combination therapies for each regimen.

During a defined treatment period, which in certain embodiments ranges from about 3 days to about 14 days from microtumor formation, the microtumors are maintained under conditions that support viability. Viability and/or growth data are acquired during the treatment period using one or more imaging-based assays. In some embodiments, fluorescence imaging with live-cell and dead-cell dyes is employed. Quantitative metrics may include changes in microtumor volume, live-cell fraction, and invasion distance into a surrounding hydrogel over time.

Based on the acquired data, a treatment response profile is determined for the tumor cells of the subject. The treatment response profile may be used to identify effective and ineffective test treatments. In some embodiments, at least one test treatment is identified as ineffective, and discontinuation of a corresponding planned therapy is recommended. A treatment regimen comprising at least one drug and a corresponding dosage is selected based on the treatment response profile.

In certain implementations, the steps of obtaining the tissue sample, generating standardized microtumors, exposing the microtumors to test treatments, and selecting the treatment regimen are completed within approximately 5 to 14 days. A report may be generated and provided to a clinician, wherein the report includes the selected treatment regimen and supporting treatment response data. This workflow enables timely, data-driven personalization of cancer therapy based on functional testing of patient-derived tumor cells.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments.

What is claimed is:

1. A multi-layer microfluidic device, comprising:
a first unit comprising
a first plurality of layers defining a first set of channels, and
a media membrane; and
a second unit comprising
a second plurality of layers defining a second set of channels, and
a chamber layer defining a hydrogel chamber, the hydrogel chamber being adapted to receive a plurality of hydrogel-encapsulated tumoroids when the first and second units are not joined to each other;
wherein the media membrane further defines the hydrogel chamber when the first and second units are joined together, the media membrane permitting the flow of a mixture of a media and a composition into the hydrogel chamber.

2. A device according to claim 1, wherein the composition includes a chemotherapeutic agent.

3. A device according to claim 1, wherein the composition includes a growth factor.

4. A device according to claim 1, further including
a reservoir defined by at least one of the first and second units;
a mixing chamber defined by at least one of the first and second units, the mixing chamber being adapted to receive the media and the composition, mix the media and the composition, and permit the mixture to flow to the hydrogel chamber; and
a cell-collection chamber defined by at least one of the first and second units.

5. A device according to claim 4, further including
a media-collection chamber defined by at least one of the first and second units; and
a terminal collection chamber defined by at least one of the first and second units.

6. A device according to claim 4, further including first follow-on collection chamber downstream of the cell collection chamber, and a second follow-on collection chamber downstream of the first follow-on collection chamber, wherein the cell collection chamber is adapted to hold cells shed from the tumoroid within about the previous day, the first follow-on collection chamber is adapted to hold cells shed from the tumoroid from about two to five days earlier, the second follow-on collection chamber is adapted to hold cells shed from the tumoroid from more than about five days earlier.

7. A device according to claim 1, wherein the hydrogel chamber includes a first cured hydrogel providing a plurality of uncured spaces, each of the uncured spaces being capable of being filled with a hydrogel-encapsulated tumoroid.

8. A device according to claim 7, wherein the hydrogel-encapsulated tumoroids are encapsulated in a second cured hydrogel.

9. A device according to claim 5, wherein the cell-collection chamber is located in a flow path between the hydrogel chamber and the terminal collection chamber;
the cell collection chamber being further defined by a cell membrane which prevents tumoroid cells from migrating from the hydrogel chamber to the terminal chamber, so that the migrating tumoroid cells are collected in the cell-collection chamber.

10. A device according to claim 9, wherein the cell membrane and the media membrane are formed from different areas of a single membrane sheet.

11. The device according to claim 8, wherein the first and second cured hydrogels are cured by different mechanisms.

12. A method for monitoring cell growth and viability, the method comprising:
providing a device comprising:
a first unit comprising a first plurality of layers defining a first set of channels;
a second unit comprising
a second plurality of layers defining a second set of channels, and
a chamber layer defining a hydrogel chamber, the hydrogel chamber being adapted to receive a plurality of hydrogel-encapsulated tumoroids when the first and second units are not joined to each other, wherein the hydrogel chamber includes a first cured hydrogel providing a plurality of uncured spaces, each of the uncured spaces being capable of being filled with a tumoroid encapsulated in a second cured hydrogel;
isolating cells from a subject or a human or animal cell line;
encapsulating the cells in the second curable hydrogel;
inserting the hydrogel-encapsulated cells into at least one of the uncured spaces in the hydrogel chamber while the first and second units are not joined to each other;
joining the first and second units together;
providing a composition;
causing the composition to flow into the hydrogel chamber and
analyzing the hydrogel-encapsulated cells in the microfluidic device.

13. The method of claim 12, further including mixing a media and the composition in a mixing chamber defined by the device.

14. The method according to claim 13, wherein the first unit includes a media membrane that further defines the hydrogel chamber when the first and second units are joined together, the media membrane permitting the flow of a mixture of a media and a composition into the hydrogel chamber.

15. The method of claim 12, wherein the composition comprises chemotherapeutic drugs, cytotoxins, antibiotics, anti-viral agents, immunomodulatory agents, and/or anti-inflammatory agents.

16. Use of the device according to claim 1 for screening the effectiveness of a composition on a tumor cell, comprising:
isolating cells from a subject or a human or animal cell line;
providing in the hydrogel chamber a first hydrogel having uncured spaces while the first and second units are not joined to each other
encapsulating the cells in a second curable hydrogel;
inserting hydrogel-encapsulated cells into the uncured spaces while the first and second units are not joined to each other;
joining the first and second units together;
providing a media;
providing a composition;
mixing the media and the composition in the mixing chamber;
causing the mixture of the composition and media to flow through the media membrane into the hydrogel chamber and
analyzing the hydrogel-encapsulated cells in the microfluidic device.

17. The use according to claim 16, wherein a cell-collection chamber is located in a flow path between the hydrogel chamber and a terminal collection chamber; and wherein the cell collection chamber is further defined by a cell membrane which prevents tumoroid cells from migrating from the hydrogel chamber to the terminal collection chamber, so that the migrating tumoroid cells are collected in the cell-collection chamber.

18. A multi-layer microfluidic device for identifying patient-specific optimal drug dosages, the device comprising:
a first inlet for introducing a dosage of a first medication at a first concentration;
a second inlet for introducing a medium without any of the first medication or with the first medication at a second concentration that is lower than the first concentration;
a plurality of outlets, at least one outlet providing a concentration of the first medication different from a concentration of another outlet;
an inlet-connecting channel fluidly connecting the first and second inlets;
a plurality of inlet wells fluidly connected to the inlet-connecting channel;
an outlet-connecting channel fluidly connecting the plurality of outlets;
a plurality of outlet wells fluidly connected to the outlet-connecting channel; and
an intermediate channel fluidly connected to at least one of the plurality of inlet wells or outlet wells.

19. A device according to claim 18, wherein each multi-layer microfluidic devices further includes a plurality of intermediate wells fluidly connected to the intermediate channel.

20. The device according to claim 19, wherein the device is adapted to receive a plurality of tumoroids, and each of the wells is adapted for receiving and containing cells.

21. A system comprising a plurality of multi-layer microfluidic devices according to claim 20.

22. The system according to claim 21, wherein the first medication in each of multi-layer microfluidic devices is different.

23. The system according to claim 21, wherein the first medication in each of multi-layer microfluidic devices is the same.

24. A multi-layer microfluidic device for evaluating therapies for a tumor, the device comprising:
an upper layer;
a lower layer; and
a middle layer positioned between the upper layer and the lower layer and that, when combined with the upper and lower layers, defines a cell chamber sized to hold an array of tumoroids, the cell chamber including a hydrogel;
wherein each of the upper, middle, and lower layers define at least five inlet channels and an outlet channel, wherein the inlet channels each provide a different medication.

25. A method of evaluating therapies for a tumor, the method comprising:
providing a microfluidic device, the microfluidic device comprising a plurality of layers, including a middle layer that, when combined with adjacent layers, defines a cell chamber in fluid communication with at least five inlet channels and an outlet channel, the cell chamber including a hydrogel, the inlet channels each providing a different medication;
placing an array of tumoroids in the cell chamber;
exposing the array of tumoroids to different combinations of medication flowing from the inlet channels; and
monitoring the reaction of the array of tumoroids to the different combinations of medications.

26. A method for evaluating the effects of different compositions on a tumor, the method comprising:
providing a microfluidic device, the microfluidic device comprising a central cell chamber and a plurality of reservoirs surrounding the central cell chamber, each of the reservoirs being in fluid communication with the central cell chamber;
placing a pre-defined tumor in the central cell chamber, such that the tumor substantially fills the central cell chamber and is adjacent each of the reservoirs;
placing a different composition in each of the reservoirs;
allowing the compositions to interact with the tumor; and
monitoring the reaction of the tumor to the different compositions.

27. The method of claim 26, further including tipping the microfluidic device to affect the interactions between a composition and the tumor.

28. The method of claim 26, wherein the compositions is a medicament, a potential carcinogen, or combinations thereof.

29. A multi-layer, multi-gel microfluidic device, comprising
a plurality of inlets, outlets, and channels in fluid communication,
a first middle layer comprising a cell-growing chamber in fluid communication with a culture medium channel, the cell-growing chamber comprising a population of tumor cells encapsulated in a cured hydrogel, and
a second middle layer comprising a hypoxic chamber positioned underneath the cell-growing chamber, such that the encapsulated tumor cells are exposed to a hypoxic environment.

30. The microfluidic device of claim 29, wherein the cell-growing chamber further comprises a second cured hydrogel, wherein the second cured hydrogel provides a plurality of uncured spaces, the uncured space containing the cured hydrogel encapsulating the tumor cells.

31. A method for evaluating the efficacy of a treatment on a subject’s tumor, the method comprising:
isolating tumor cells from a subject’s tumor;
encapsulating the tumor cells in a curable hydrogel;
inserting the cells in a multi-layer, multi-gel microfluidic device comprising
a plurality of inlets, outlets, and channels in fluid communication,
a first middle layer comprising a cell-growing chamber in fluid communication with a culture medium channel, the cell-growing chamber comprising a population of tumor cells encapsulated in a cured hydrogel, and
a second middle layer comprising a hypoxic chamber underneath the cell-growing chamber, such that the encapsulated tumor cells are exposed to a hypoxic environment;
incubating the encapsulated tumor cells in the microfluidic device under conditions favorable for tumor cell growth for a period of time;
contacting the encapsulated tumor cells with one or more drugs for a period of time;
analyzing changes or viability in the encapsulated tumor cells after the period of time;
evaluating the efficacy of the drug on the encapsulated tumor cells; and
initiating, maintaining, or modifying treatment and dosage thereof of the subject from whom the tumor cells were isolated.
